(19) United States
(12) Patent Application Publication
(10) Pub. No.: US 2023/0135524 A1
(43) Pub. Date: May 4, 2023

(54) ARYL HYDANTOIN HETEROCYCLES AND METHODS OF USE

(71) Applicants: The United States of America,as represented by the Secretary,Department of Health and Human Services, Bethesda, MD (US); Georgia Tech Research Corporation, Atlanta, GA (US)

(72) Inventors: Berkley E. Gryder, Wickliffe, OH (US); Adegboyega K. Oyelere, Marietta, GA (US); Subhasish Tapadar, Atlanta, GA (US); Jonathan D. Strope, Hyattsville, MD (US); William Douglas Figg, Sr., Tierra Verde, FL (US)

(73) Assignees: The United States of America,as represented by the Secretary,Department of Health and Human Services, Bethesda, MD (US); Georgia Tech Research Corporation, Atlanta, GA (US)

(21) Appl. No.: 17/794,212

(22) PCT Filed: Jan. 20, 2021
(86) PCT No.: PCT/US2021/014176

§ 371 Date: Jul. 20, 2022

Related U.S. Application Data
(60) Provisional application No. 62/963,959, filed on Jan. 21, 2020.

(51) Int. Cl. C07D 403/10; C07D 403/06; C07D 401/14

(52) U.S. Cl. CPC C07D 403/10; C07D 403/06; C07D 401/14

(57) ABSTRACT

Disclosed is a compound of formula (I) in which R¹, R², R³, X¹, X², X^{2′}, X³, X⁴, ring A, m, n, and o are as described herein. The compound of formula (I) is useful for treating a disorder associated with androgen receptor malfunction, such as a hyperproliferative disorder, in a subject in need thereof.

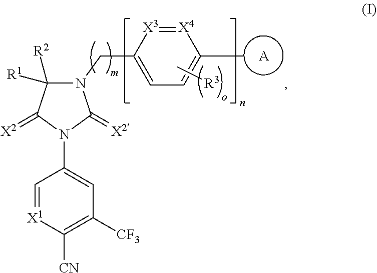

### CROSS-REFERENCE TO A RELATED APPLICATION

This patent application claims the benefit of U.S. Provisional Patent Application No. 62/963,959, filed Jan. 21, 2020, which is incorporated by reference for all purposes.

### BACKGROUND OF THE INVENTION

Prostate cancer remains the most common form of cancer among all males in the United States with black men at highest risk (Jemal et al., CA Cancer J Clin, 2009, 59(4), 225-249). Prostate cancer is also the second leading cause of cancer related deaths in the United States among men, largely due to the progressively treatment-resistant nature of the disease. Treatment options for early stage prostate cancer commonly involve various combinations of watchful waiting, radical prostatectomy, radiation therapy, and androgen-deprivation therapy (ADT) (Pirtskhalaishvili et al., Cancer Practice, 2001, 9 (6), 295-306.). Prostate cancer is dependent upon androgen hormone steroids such as dihydrotestosterone (DHT) for sustaining and promoting growth. The androgen hormone steroids bind to the androgen receptor (AR) and localize to the nucleus where it forms a complex that up-regulates the transcription of critical genes. ADT is accomplished by either (i) administering antagonist to the AR that blocks androgen ligands, or (ii) by castration, in order to reduce the amount of testosterone available. Often both methods of ADT are used. However, the disease frequently advances to the much more lethal castration-resistant prostate cancer (CRPC), becoming resistant to these therapies by overexpressing ARs (Chen et al., Nature Medicine, 2004, 10(1), 33-39; and Papatsoris et al., Current Medicinal Chemistry, 2005, 12(3), 277-296). The expression levels of AR are about six-fold higher in CRPC as compared to hormone-sensitive prostate cancer (Linja et al., Cancer Res., 2001, 61(9), 3550-3555). Options currently available for CRPC are supportive care, salvage endocrine manipulations, radiotherapy, radioactive isotopes, bisphosphonates, and chemotherapy (Lara et al., Cancer Investigation, 1999, 17(2), 137-144). However, these options are not curative. The understanding that AR over-expression is one of the major causes of hormone refractory prostate cancer, and the dependency of the growth of the hormone refractory prostate on the binding of AR ligands, suggest that AR is a viable target for this form of malignancy. The preference of anti-androgens as agents for prostate cancer therapy is predicated on the selectivity and fewer side effects of these agents. Use of anti-androgens has been proposed in the patent literature. However, the anti-androgens in common clinical use, such as bicalutamide (Casodex), have curative effects only on hormone sensitive prostate cancer and not on hormone refractory prostate cancer. The lack of the activity of most anti-androgens against refractory prostate cancer is partly due to their weak antagonist activities and strong agonist activities when AR is over-expressed as in refractory prostate cancer.

Thus, there remains an unmet need to provide compounds that inhibit the androgen receptor with excellent efficacy for treating a disorder associated with androgen receptor malfunction, such as prostate cancer.

### BRIEF SUMMARY OF THE INVENTION

The invention provides a compound of formula (I)

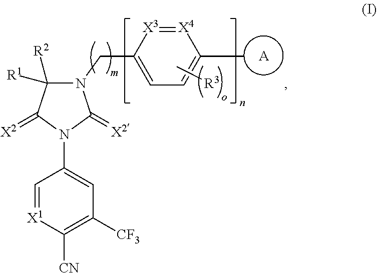

in which R¹, R², R³, X¹, X², X², X³, X⁴, ring A, m, n, and o are described herein.

Further provided is a method of treating a disorder associated with androgen receptor malfunction, such as a hyperproliferative disorder, in a subject with an effective amount of the compound of formula (I) or a pharmaceutically acceptable salt thereof.

### BRIEF DESCRIPTION OF THE SEVERAL VIEWS OF THE DRAWINGS

FIG. 1 is a chemical scheme illustrating an example synthesis of arylhydantoin alkynes.

FIG. 2 is a chemical scheme illustrating an example synthesis of arylhydantoin triazoles.

FIG. 3 is a chemical scheme illustrating an example synthesis of reverse triazoles.

FIG. 4 is a chemical scheme illustrating a synthesis of aryl hydantoin forward triazoles with polar substituents.

FIG. 5 shows the AR binding affinity measured by competition with radiolabeled [3H]mibolerone with increasing concentrations of ligands 14a, 15a, and 16 compared to enzalutamide.

FIG. 6A-C show AR transcriptional activity remaining after 24 hours of dosing with 10 μM, as tested by ARE-luciferase assay in HEK293 cells with compounds 6, 10, 13, 14a, 15a, 16a, and bicalutamide (FIG. 6A), compounds 15a-e and bicalutamide (FIG. 6B), and compound 15a and enzalutamide (FIG. 6C). 100% is set to the activity with 200 μM testosterone, and 0% is set to the basal level (no ligand).

FIG. 7 shows the antagonist or inverse agonist activity of 20, 20a-e, bicalutamide, enzalutamide, and cyanonilutamide.

FIG. 8A-B show the ability for 14a, 14b, 15a, 15b, 16a, enzalutamide, and bicalutamide to kill LNCaP cells at varying doses.

FIG. 9A-B show the effect of 19c on the growth of LNCaP-AR cells (FIG. 9A) compared to enzalutamide (FIG. 9B).

FIG. 10A-H are dose response curves for 16a (FIGS. 10A and 10E) 19a (FIGS. 10B and 10F), enzalutamide (FIGS. 10C and 10G), and bicalutamide (FIGS. 10D and 10H) against AR+ prostate cancer cells lines: LNCaP-AR (FIG. 10A-D) and LAPC4 (FIG. 10E-H). The graph markers denote: ● is 0 hours, ▪ is 24 hours, ▴ is 48 hours, ▾ is 72 hours, and ♦ is 96 hours.

FIG. 11 is a graph of drug concentration (nM) versus percent inhibition for 19d and testosterone.

FIG. 12 is a graph of the drug concentrations of 15a and enzalutamide, as measured by days of measurement versus tumor volume (mm³).

FIG. 13 is a graph of log P (octanol:water partition) versus molecular mass (Da).

FIG. 14A-B are graphs of time (hours) versus plasma concentration (ng/mL) for intravenous (IV) (FIG. 14A) and oral (PO) (FIG. 14B) administration of 15a and 15n over 12 hours. Error bars represent the standard deviation in ng/mL from three independent mice per time point.

FIG. 15 is a table of a radioligand binding study showing percent inhibition of various receptors, the concentration of drug producing 50% inhibition (IC₅₀), the inhibition constant (Kᵢ), and n_{H} at different concentrations of 15n.

FIG. 16A-B are a Gene Set Enrichment Analysis for AR gene pathways (FIG. 16A) and MYC gene pathways (FIG. 16B) in LAMP cells (LNCaP cells engineered with CRISPR to have amplification of the AR enhancer) treated with either 15n or dimethylsulfoxide (DMSO) for 48 hours, followed by RNA-seq.

FIG. 17 illustrates the rank order of gene expression for cluster 4 to cluster 1 showing transcription factor (TF) priority switches during relapse.

FIG. 18 shows AR-enhancer amplification by CRISPR knock-in to LNCaP cells to create LAMP cells.

FIG. 19 shows the expression of the top 3 candidate master TFs found in relapsed mCRPC (ATF4, XPB1 and the AR) in LAMP cells treated with DMSO, enzalutamide, or 15n for 48 hours.

FIG. 20 is a graph of growth reduction kinetics in LAMP cells treated with enzalutamide or 15n at 15 μM and 30 μM, compared to a DMSO control.

FIG. 21 is a model of androgen deprivation therapy (ADT), enzalutamide resistance by AR amplification, and overcoming it with 15a/n.

FIG. 22 is a graph of tumor volume on day 17 as a percent change from the baseline.

FIG. 23 is a graph of days on treatment versus percent relative growth for vehicle (●), enzalutamide (▪), and 15n (▴).

### DETAILED DESCRIPTION OF THE INVENTION

The invention provides aryl hydantoin-based heterocyclic compounds that are inverse agonists of the androgen receptor with improved efficacy and methods of making and using thereof. Accordingly, the invention provides a compound of formula (I):

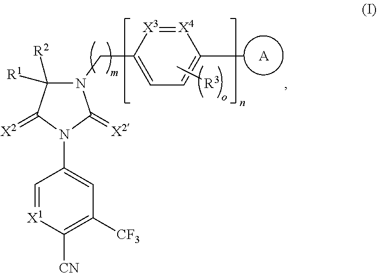

wherein

R¹ and R² are the same or different and each is alkyl,

X¹ is CH or N,

X² and X² are the same or different and each is O or S,

X³ and X⁴ are the same or different and each is CR³ or N,

R³ is hydrogen, halo, hydroxy, alkyl, or alkoxy,

ring A is selected from the group consisting of

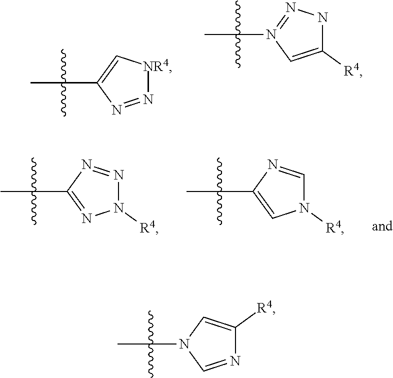

R⁴ is hydrogen, alkyl, cycloalkyl, heterocycloalkyl, aryl, or heteroaryl, wherein the alkyl, cycloalkyl, heterocycloalkyl, aryl, or heteroaryl is optionally substituted,

m is 0-8, n is 0 or 1, such that 1≤m+n≤9, and o is 0-2,

provided that when X¹ is CH, X³ and X⁴ are each CR³, ring A is

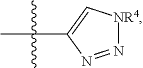

R⁴ is hydrogen or alkyl, n is 1, and o is 0-2, then at least one R³ is not hydrogen,
or a pharmaceutically acceptable salt thereof.

In some embodiments of formula (I), compounds in which X¹ is CH, ring A is

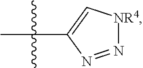

n is 1, and R³ is hydrogen are excluded.

In any of the aspects described herein, R¹ and R² are the same or different and can be alkyl (e.g., C₁₋₈ alkyl), such as methyl, ethyl, isopropyl, or n-butyl. In some aspects, R¹ and R² are each methyl.

In any of the aspects described herein, X¹ is N. In other aspects herein, X¹ is CH.

In any of the foregoing aspects, X³ and X⁴ are each CR³, in which R³ is hydrogen, halo (e.g., F, Cl, Br, or I), hydroxy, alkyl (e.g., C₁₋₈ alkyl), or alkoxy.

In any of the aspects described herein, ring A is

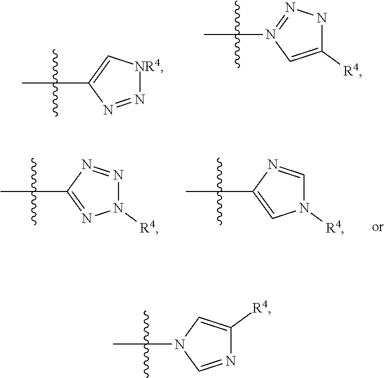

and preferably, ring A is

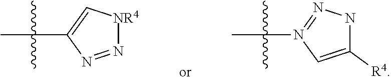

In some aspects, the compound of formula (I) is a compound of formula (Ia):

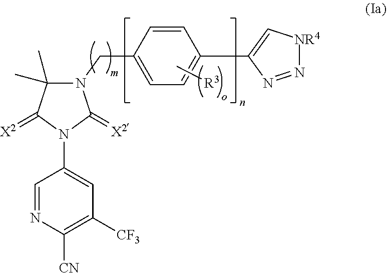

wherein

X² and X² are the same or different and each is O or S,

R³ is hydrogen, halo, hydroxy, alkyl, or alkoxy,

R⁴ is hydrogen, alkyl, cycloalkyl, heterocycloalkyl, aryl, or heteroaryl, wherein the

alkyl, cycloalkyl, heterocycloalkyl, aryl, or heteroaryl is optionally substituted,

m is 0-8, n is 0 or 1, such that 1≤m+n≤9, and o is 0-2,

or a pharmaceutically acceptable salt thereof.

In any of the foregoing aspects of the compound of formula (I) or (Ia), preferably at least one R³ moiety is a halo, such as fluoro (e.g., 2-fluoro or 3-fluoro).

In any of the foregoing aspects of the compound of formula (I) or (Ia), X² and X² are the same or different. In some preferred aspects set forth herein, X² is O and X² is S or X² and X² are both O.

In any of the foregoing aspects of the compound of formula (I) or (Ia), m is 0 or any integer from 1-8 (i.e., 1, 2, 3, 4, 5, 6, 7, or 8). In some aspects, m is 0, 1, or 2; preferably m is 1.

In any of the foregoing aspects of the compound of formula (I) or (Ia), n is 0 or 1. In some aspects, n is 1. When m is 0, n must be 1.

In any of the foregoing aspects of the compound of formula (I) or (Ia), R⁴ is hydrogen or alkyl that is optionally substituted. In some preferred aspects, the alkyl is substituted with one or more substituents selected from hydroxyl, cyano, amino, alkylamino, dialkylamino, halo, nitro, carboxy, amido, cycloalkyl, heterocycloalkyl, aryl, and heteroaryl. Preferably, R⁴ is hydrogen, methyl, ethyl, n-propyl, n-butyl, hydroxymethyl, mono- or dihydroxy-substituted ethyl, mono- or dihydroxy-substituted propyl, mono- or dihydroxy-substituted butyl, mono- or dihydroxy-substituted pentyl, cyano-substituted ethyl, amino-substituted propyl, amino-substituted butyl, amino-substituted pentyl, carboxymethyl (—CH₂C(O)OH), amidomethyl (—CH₂C(O)NH₂), moropholinylethyl, or phenyl. In any of the foregoing aspects of the compound of formula (I) or (Ia), R⁴ preferably is hydrogen.

Exemplary compounds of the invention include, wherein R³ is not hydrogen:

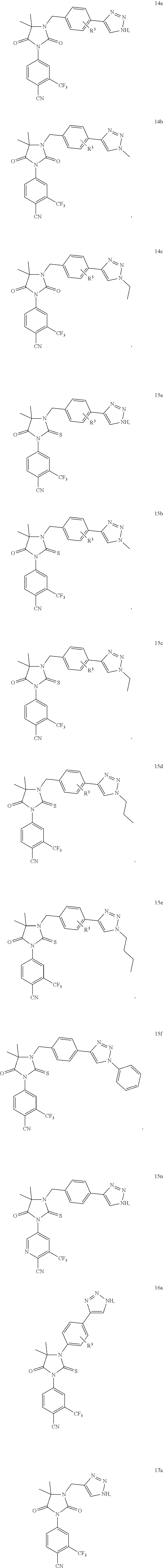

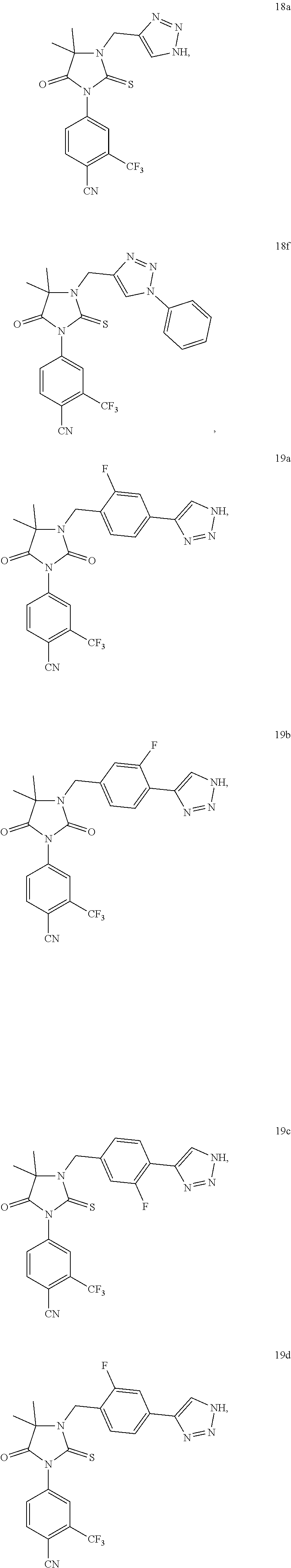 [SMILES: CC1(C)C(=O)N(c2ccc(C#N)c(C(F)(F)F)c2)C(=S)N1Cc1c[nH]nn1 ; CC1(C)C(=O)N(c2ccc(C#N)c(C(F)(F)F)c2)C(=S)N1Cc1cn(-c2ccccc2)nn1 ; CC1(C)C(=O)N(c2ccc(C#N)c(C(F)(F)F)c2)C(=O)N1Cc1ccc(-c2c[nH]nn2)cc1F ; CC1(C)C(=O)N(c2ccc(C#N)c(C(F)(F)F)c2)C(=O)N1Cc1ccc(-c2c[nH]nn2)c(F)c1 ; CC1(C)C(=O)N(c2ccc(C#N)c(C(F)(F)F)c2)C(=S)N1Cc1ccc(-c2c[nH]nn2)c(F)c1 ; CC1(C)C(=O)N(c2ccc(C#N)c(C(F)(F)F)c2)C(=S)N1Cc1ccc(-c2c[nH]nn2)cc1F]

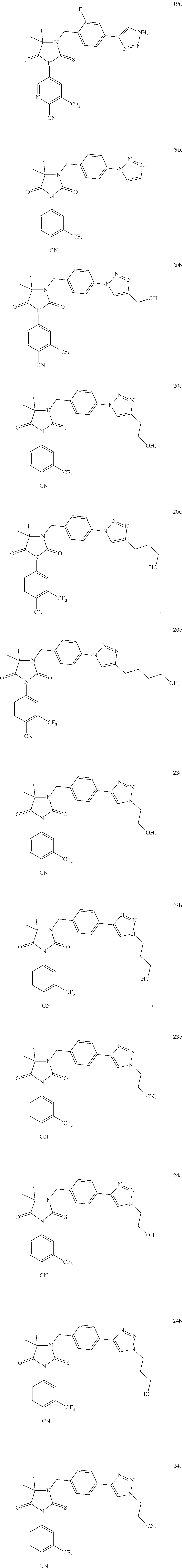

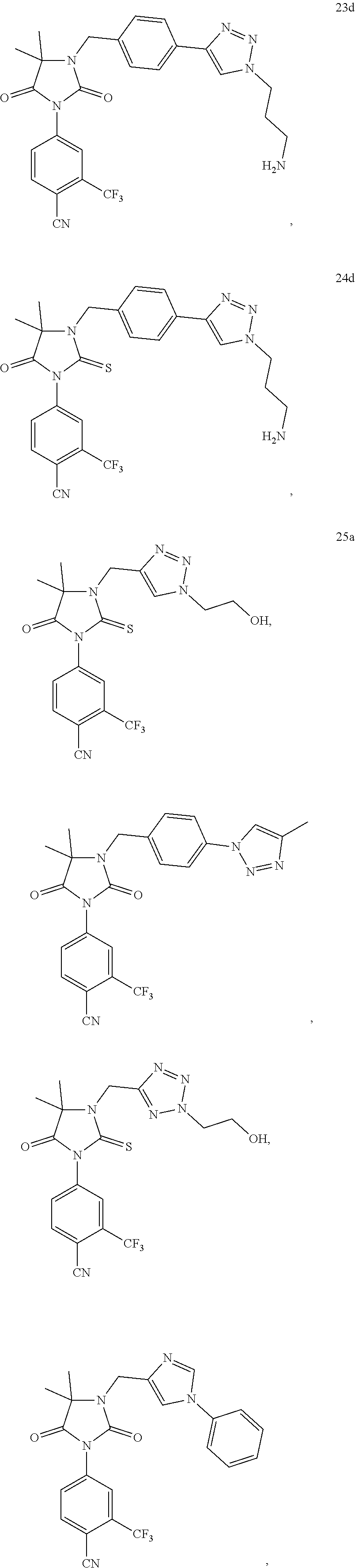 [SMILES: CC1(C)C(=O)N(c2ccc(C#N)c(C(F)(F)F)c2)C(=O)N1Cc1ccc(-c2cn(CCCN)nn2)cc1 ; CC1(C)C(=O)N(c2ccc(C#N)c(C(F)(F)F)c2)C(=S)N1Cc1ccc(-c2cn(CCCN)nn2)cc1 ; CC1(C)C(=O)N(c2ccc(C#N)c(C(F)(F)F)c2)C(=S)N1Cc1cn(CCO)nn1 ; Cc1cn(-c2ccc(CN3C(=O)N(c4ccc(C#N)c(C(F)(F)F)c4)C(=O)C3(C)C)cc2)nn1 ; CC1(C)C(=O)N(c2ccc(C#N)c(C(F)(F)F)c2)C(=S)N1Cc1nnn(CCO)n1 ; CC1(C)C(=O)N(c2ccc(C#N)c(C(F)(F)F)c2)C(=O)N1Cc1cn(-c2ccccc2)cn1]

[chemical structure]

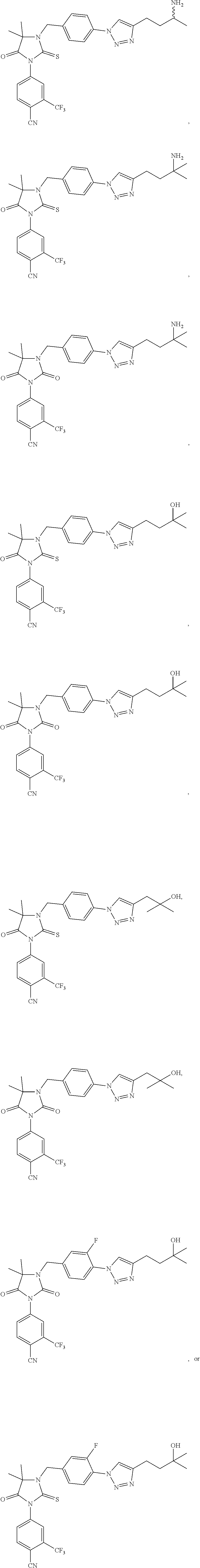

or a pharmaceutically acceptable salt thereof.

In some preferred aspects, the compound of formula (I) is

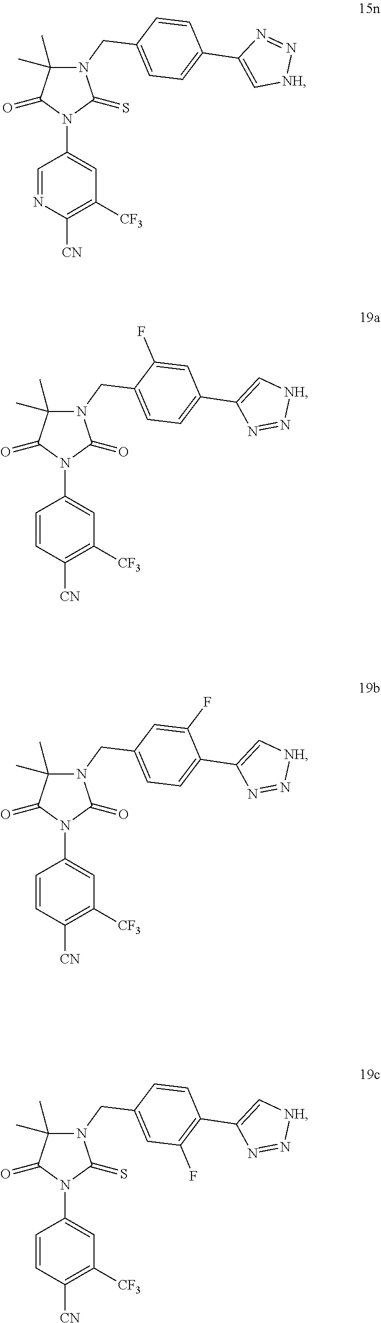 [SMILES: CC1(C)C(=O)N(c2cnc(C#N)c(C(F)(F)F)c2)C(=S)N1Cc1ccc(-c2c[nH]nn2)cc1 ; CC1(C)C(=O)N(c2ccc(C#N)c(C(F)(F)F)c2)C(=O)N1Cc1ccc(-c2c[nH]nn2)cc1F ; CC1(C)C(=O)N(c2ccc(C#N)c(C(F)(F)F)c2)C(=O)N1Cc1ccc(-c2c[nH]nn2)c(F)c1 ; CC1(C)C(=O)N(c2ccc(C#N)c(C(F)(F)F)c2)C(=S)N1Cc1ccc(-c2c[nH]nn2)c(F)c1]

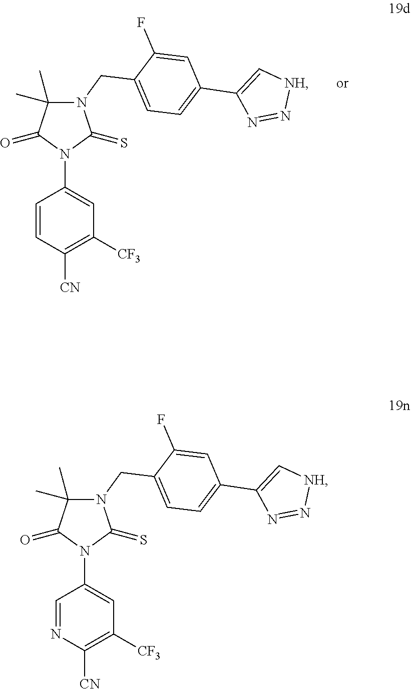 [SMILES: CC1(C)C(=O)N(c2ccc(C#N)c(C(F)(F)F)c2)C(=S)N1Cc1ccc(-c2c[nH]nn2)cc1F ; CC1(C)C(=O)N(c2cnc(C#N)c(C(F)(F)F)c2)C(=S)N1Cc1ccc(-c2c[nH]nn2)cc1F]

or a pharmaceutically acceptable salt thereof. In an especially preferred aspect, the compound of formula (I) is 15n.

In any of the aspects above, the term “alkyl” implies a straight-chain or branched alkyl substituent containing from, for example, from about 1 to about 8 carbon atoms, e.g., from about 1 to about 6 carbon atoms. Examples of alkyl group include methyl, ethyl, n-propyl, isopropyl, n-butyl, sec-butyl, isobutyl, tert-butyl, n-pentyl, isopentyl, n-hexyl, and the like. This definition also applies wherever “alkyl” occurs as part of a group, such as, e.g., in alkoxy, hydroxyalkyl, haloalkyl (e.g., monohaloalkyl, dihaloalkyl, and trihaloalkyl), cyanoalkyl, aminoalkyl, alkylamino, dialkylamino, arylalkyl, etc. The alkyl can be substituted or unsubstituted, as described herein. Even in instances in which the alkyl is an alkylene chain (e.g., —(CH₂)ₙ—), the alkyl group can be substituted or unsubstituted.

In any of the aspects above, the term “cycloalkyl,” as used herein, means a cyclic alkyl moiety containing from, for example, 3 to 6 carbon atoms or from 5 to 6 carbon atoms. Examples of such moieties include cyclopropyl, cyclobutyl, cyclopentyl, cyclohexyl, and the like. The cycloalkyl can be substituted or unsubstituted, as described herein.

In any of the aspects above, the term “hydroxyl” refers to the group —OH.

In any of the aspects above, the term “alkoxy” embraces a linear or branched alkyl group that is attached to a divalent oxygen. The alkyl group of the alkoxy is as described above for alkyl.

In any of the aspects above, the term “halo” refers to a halogen selected from fluorine, chlorine, bromine, and iodine.

In any of the aspects above, the term “aryl” refers to a mono, bi, or tricyclic carbocyclic ring system having one, two, or three aromatic rings, for example, phenyl, naphthyl, anthracenyl, or biphenyl. The term “aryl” refers to an unsubstituted or substituted aromatic carbocyclic moiety, as commonly understood in the art, and includes monocyclic and polycyclic aromatics such as, for example, phenyl, biphenyl, naphthyl, anthracenyl, pyrenyl, and the like. An aryl moiety generally contains from, for example, 6 to 30 carbon atoms, from 6 to 18 carbon atoms, from 6 to 14 carbon atoms, or from 6 to 10 carbon atoms. It is understood that the term aryl includes carbocyclic moieties that are planar and comprise 4n+2 π electrons, according to Hickel's Rule, wherein n=1, 2, or 3. The aryl can be substituted or unsubstituted, as described herein.

In any of the aspects above, the term “heteroaryl” refers to aromatic 5 or 6 membered monocyclic groups, 9 or 10 membered bicyclic groups, and 11 to 14 membered tricyclic groups which have at least one heteroatom (O, S, or N) in at least one of the rings. Each ring of the heteroaryl group containing a heteroatom can contain one or two oxygen or sulfur atoms and/or from one to four nitrogen atoms provided that the total number of heteroatoms in each ring is four or less and each ring has at least one carbon atom. The fused rings completing the bicyclic and tricyclic groups may contain only carbon atoms and may be saturated, partially saturated, or unsaturated. The nitrogen and sulfur atoms may optionally be oxidized, and the nitrogen atoms may optionally be quaternized. Heteroaryl groups which are bicyclic or tricyclic must include at least one fully aromatic ring but the other fused ring or rings may be aromatic or non-aromatic. The heteroaryl group can be attached at any available nitrogen or carbon atom of any ring. Illustrative examples of heteroaryl groups are pyridinyl, pyridazinyl, pyrimidyl, pyrazinyl, benzimidazolyl, triazinyl, imidazolyl, (1,2,3)- and (1,2,4)-triazolyl, pyrazinyl, tetrazolyl, furanyl, benzofuranyl, pyrrolyl, thienyl, isothiazolyl, thiazolyl, isoxazolyl, and oxadiazolyl. The heteroaryl can be substituted or unsubstituted, as described herein.

The term “heterocycloalkyl” means a stable, saturated, or partially unsaturated monocyclic, bicyclic, and spiro ring system containing 3 to 7 ring members of carbon atoms and other atoms selected from nitrogen, sulfur, and/or oxygen. In an aspect, a heterocycloalkyl is a 5, 6, or 7-membered monocyclic ring and contains one, two, or three heteroatoms selected from nitrogen, oxygen, and sulfur. The heterocycloalkyl can be attached to the parent structure through a carbon atom or through any heteroatom of the heterocycloalkyl that results in a stable structure. Examples of such heterocycloalkyl rings are isoxazolyl, thiazolinyl, imidazolidinyl, piperazinyl, homopiperazinyl, pyrrolyl, pyrrolinyl, pyrazolyl, pyranyl, piperidyl, oxazolyl, and morpholinyl. The heterocycloalkyl can be substituted or unsubstituted, as described herein.

In other aspects, any substituent that is not hydrogen (e.g., C₁-C₈ alkyl, C₃-C₆ cycloalkyl, aryl, arylalkyl, heteroaryl, heteroarylalkyl, heterocycloalkyl, or heterocycloalkylalkyl) can be an optionally substituted moiety. The substituted moiety typically comprises at least one substituent (e.g., 1, 2, 3, 4, 5, 6, etc.) in any suitable position (e.g., 1-, 2-, 3-, 4-, 5-, or 6-position, etc.). When an aryl group is substituted with a substituent, e.g., halo, amino, alkyl, OH, alkoxy, and others, the aromatic ring hydrogen is replaced with the substituent and this can take place in any of the available hydrogens, e.g., 2, 3, 4, 5, and/or 6-position wherein the 1-position is the point of attachment of the aryl group in the compound of the present invention. Suitable substituents include, e.g., halo, alkyl, alkenyl, alkynyl, hydroxy, nitro, cyano, amino, alkylamino, dialkylamino, alkoxy, aryloxy, aralkoxy, carboxyl, carboxyalkyl, carboxyalkyloxy, amido, alkylamido, haloalkylamido, aryl, heteroaryl, and heterocycloalkyl, as described herein.

In any of the aspects above, whenever a range of the number of atoms in a structure is indicated (e.g., a C₁₋₈, C₁₋₆, or C₁₋₄ alkyl, cycloalkyl, etc.), it is specifically contemplated that any sub-range or individual number of carbon atoms falling within the indicated range also can be used. Thus, for instance, the recitation of a range of 1-8 carbon atoms (e.g., C₁-C₈), 1-6 carbon atoms (e.g., C₁-C₆), 1-4 carbon atoms (e.g., C₁-C₄), 1-3 carbon atoms (e.g., C₁-C₃), or 2-8 carbon atoms (e.g., C₂-C₈) as used with respect to any chemical group (e.g., alkyl, cycloalkyl, etc.) referenced herein encompasses and specifically describes 1, 2, 3, 4, 5, 6, 7, and/or 8 carbon atoms, as appropriate, as well as any sub-range thereof (e.g., 1-2 carbon atoms, 1-3 carbon atoms, 1-4 carbon atoms, 1-5 carbon atoms, 1-6 carbon atoms, 1-7 carbon atoms, 1-8 carbon atoms, 2-3 carbon atoms, 2-4 carbon atoms, 2-5 carbon atoms, 2-6 carbon atoms, 2-7 carbon atoms, 2-8 carbon atoms, 3-4 carbon atoms, 3-5 carbon atoms, 3-6 carbon atoms, 3-7 carbon atoms, 3-8 carbon atoms, 4-5 carbon atoms, 4-6 carbon atoms, 4-7 carbon atoms, 4-8 carbon atoms, etc., as appropriate).

The subscript “m” represents the number of methylene repeat units. The subscript “n” represents the number of repeat units of the aromatic unit (0 or 1). In view of the formula “1≤m+n≤9,” there must be at least one methylene repeat unit or one aromatic unit present in the compound of formula (I). The subscript “o” represents the number of substituents R³, in which each substituent, e.g., R³, can be the same or different. When m, n, or o is 0, then the corresponding moiety, i.e., methylene group, aromatic unit, or R³, is not present in the compound of formula (I) or (Ia).

In any of the aspects above, the phrase “salt” or “pharmaceutically acceptable salt” is intended to include nontoxic salts synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. For example, an inorganic acid (e.g., hydrochloric acid, sulfuric acid, phosphoric acid, or hydrobromic acid), an organic acid (e.g., oxalic acid, malonic acid, citric acid, fumaric acid, lactic acid, malic acid, succinic acid, tartaric acid, acetic acid, trifluoroacetic acid, gluconic acid, ascorbic acid, methylsulfonic acid, or benzylsulfonic acid), an inorganic base (e.g., sodium hydroxide, potassium hydroxide, calcium hydroxide, magnesium hydroxide, or ammonium hydroxide), an organic base (e.g., methylamine, diethylamine, triethylamine, triethanolamine, ethylenediamine, tris(hydroxymethyl)methylamine, guanidine, choline, or cinchonine), or an amino acid (e.g., lysine, arginine, or alanine) can be used. Generally, nonaqueous media such as ether, ethyl acetate, ethanol, isopropanol, or acetonitrile are typical. Lists of suitable salts are found in Remington's Pharmaceutical Sciences, 18th ed., Mack Publishing Company, Easton, Pa., 1990, p. 1445, and Journal of Pharmaceutical Science, 66, 2-19 (1977). For example, they can be a salt of an alkali metal (e.g., sodium or potassium), alkaline earth metal (e.g., calcium), or ammonium of salt.

The compounds described herein can have one or more chiral centers, and thus, exist as one or more stereoisomers. Such stereoisomers can exist as a single enantiomer, a mixture of diastereomers, or a racemic mixture. As used herein, the term “stereoisomers” refers to compounds made up of the same atoms having the same bond order but having different three-dimensional arrangements of atoms that are not interchangeable. The three-dimensional structures are called configurations. As used herein, the term “enantiomers” refers to two stereoisomers which are non-superimposable mirror images of one another. As used herein, the term “optical isomer” is equivalent to the term “enantiomer”. As used herein the term “diastereomer” refers to two stereoisomers which are not mirror images but also not superimposable. The terms “racemate,” “racemic mixture,” or “racemic modification” refer to a mixture of equal parts of enantiomers. The term “chiral center” refers to a carbon atom to which four different groups are attached. Choice of the appropriate chiral column, eluent, and conditions necessary to effect separation of a pair of enantiomers is well known to one of ordinary skill in the art using standard techniques (see e.g. Jacques et al., “Enantiomers, Racemates, and Resolutions,” John Wiley and Sons, Inc. 1981).

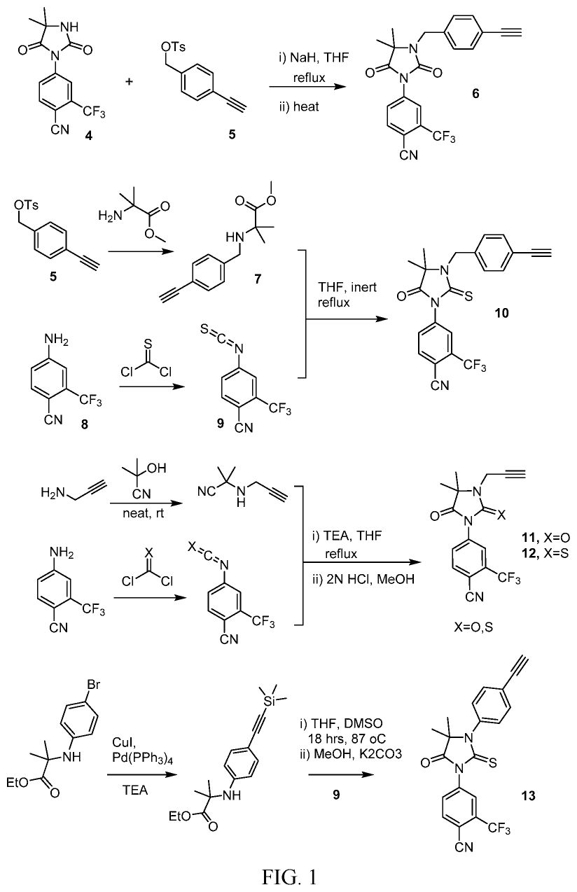

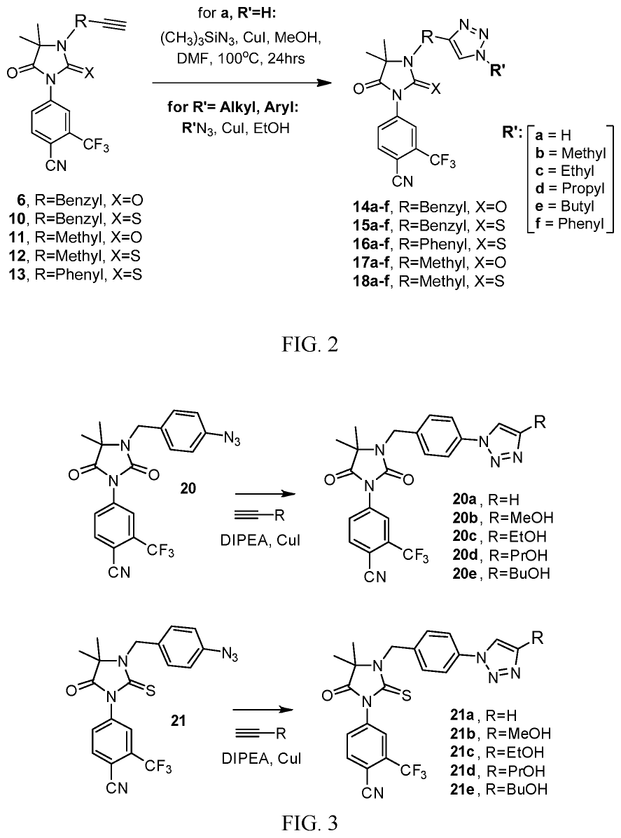

A compound of formula (I) or (Ia) can be prepared by any suitable method, including the methods described herein. An example synthesis of arylhydantoin alkynes is shown in FIG. 1. An example synthesis of arylhydantoin triazoles is shown in FIG. 2. An example synthesis of reverse triazoles is shown in FIG. 3. A synthesis of aryl hydantoin forward triazoles with polar substituents is shown in FIG. 4.

The methods described herein comprise using a compound of formula (I) or (Ia) or a pharmaceutically acceptable salt thereof in the form of a pharmaceutical composition. In particular, a pharmaceutical composition will comprise (i) at least one compound of formula (I) or (Ia) or a pharmaceutically acceptable salt thereof and (ii) a pharmaceutically acceptable carrier. The pharmaceutically acceptable excipients described herein, for example, vehicles, adjuvants, carriers or diluents, are well-known to those who are skilled in the art and are readily available to the public. Typically, the pharmaceutically acceptable carrier is one that is chemically inert to the active compounds and one that has no detrimental side effects or toxicity under the conditions of use.

The pharmaceutical compositions can be administered as oral, sublingual, transdermal, subcutaneous, topical, absorption through epithelial or mucocutaneous linings, intravenous, intranasal, intraarterial, intramuscular, intratumoral, peritumoral, interperitoneal, intrathecal, rectal, vaginal, or aerosol formulations. In some aspects, the pharmaceutical composition is administered orally (e.g., enteral) or intravenously (e.g., parenteral).

In accordance with any of the aspects, the compound of formula (I) or (Ia) or a pharmaceutically acceptable salt thereof can be administered orally to a subject in need thereof. Formulations suitable for oral administration can include (a) liquid solutions, such as an effective amount of the compound dissolved in diluents, such as water, saline, or orange juice and include an additive, such as cyclodextrin (e.g., α-, β-, or γ-cyclodextrin, hydroxypropyl cyclodextrin) or polyethylene glycol (e.g., PEG400); (b) capsules, sachets, tablets, lozenges, and troches, each containing a predetermined amount of the active ingredient, as solids or granules; (c) powders; (d) suspensions in an appropriate liquid; and (e) suitable emulsions and gels. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and cornstarch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compound of formula (I) or (Ia) or a salt thereof can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene-polypropylene copolymers, (d) amphoteric detergents such as, for example, alkyl-beta-aminopropionates, and 2-alkyl-imidazoline quaternary ammonium salts, and (3) mixtures thereof.

The parenteral formulations will typically contain from about 0.5 to about 25% by weight of the active compound in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

The compounds may be made into injectable formulations. The requirements for effective pharmaceutical carriers for injectable compositions are well known to those of ordinary skill in the art. See Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986).

The dose administered to the subject, particularly human and other mammals, in accordance with the present invention should be sufficient to affect the desired response. One skilled in the art will recognize that dosage will depend upon a variety of factors, including the age, condition or disease state, predisposition to disease, genetic defect or defects, and body weight of the subject. The size of the dose will also be determined by the route, timing and frequency of administration as well as the existence, nature, and extent of any adverse side-effects that might accompany the administration of a particular compound and the desired effect. It will be appreciated by a person of skill in the art that various conditions or disease states may require prolonged treatment involving multiple administrations.

The inventive methods comprise administering an effective amount of a compound of formula (I) or (Ia) or a pharmaceutically acceptable salt thereof. An “effective amount” means an amount sufficient to show a meaningful benefit in an individual, e.g., promoting at least one aspect of tumor cell cytotoxicity (e.g., inhibition of growth, inhibiting survival of a cancer cell, reducing proliferation, reducing size and/or mass of a tumor (e.g., solid tumor)), or treatment, healing, prevention, delay of onset, halting, or amelioration of other relevant medical condition(s) associated with a particular cancer. The meaningful benefit observed in the patient can be to any suitable degree (10, 20, 30, 40, 50, 60, 70, 80, 90% or more). In some aspects, one or more symptoms of the cancer are prevented, reduced, halted, or eliminated subsequent to administration of a compound of formula (I), including a compound of formula (Ia), or a pharmaceutically acceptable salt thereof, thereby effectively treating the cancer to at least some degree.

Effective amounts may vary depending upon the biological effect desired in the individual, condition to be treated, and/or the specific characteristics of the compound of formula (I), including a compound of formula (Ia), or a pharmaceutically acceptable salt thereof, and the individual. In this respect, any suitable dose of the compound of formula (I) or a pharmaceutically acceptable salt thereof can be administered to the subject (e.g., human), according to the type of cancer to be treated. Various general considerations taken into account in determining the “effective amount” are known to those of skill in the art and are described, e.g., in Gilman et al., eds., Goodman And Gilman's: The Pharmacological Bases of Therapeutics, 8th ed., Pergamon Press, 1990; and Remington's Pharmaceutical Sciences, 17th Ed., Mack Publishing Co., Easton, Pa., 1990, each of which is herein incorporated by reference. The dose of the compound of formula (I) or (Ia) or a pharmaceutically acceptable salt thereof desirably is in an amount that enables the compound to act as an inverse agonist of AR, e.g., about 0.1 mg per kilogram (kg) of the body weight of the subject (mg/kg) to about 400 mg/kg (e.g., about 0.75 mg/kg, about 5 mg/kg, about 30 mg/kg, about 75 mg/kg, about 100 mg/kg, about 200 mg/kg, or about 300 mg/kg). In another aspect, the dose of the compound of formula (I) or (Ta) or salt thereof comprises about 0.5 mg/kg to about 300 mg/kg (e.g., about 0.75 mg/kg, about 5 mg/kg, about 50 mg/kg, about 100 mg/kg, or about 200 mg/kg), about 10 mg/kg to about 200 mg/kg (e.g., about 25 mg/kg, about 75 mg/kg, or about 150 mg/kg), or about 50 mg/kg to about 100 mg/kg (e.g., about 60 mg/kg, about 70 mg/kg, or about 90 mg/kg).

In an aspect, a compound formula (I), including a compound of formula (Ia), or a pharmaceutically acceptable salt thereof is an inverse agonist of androgen receptor (AR). AR overexpression is one of the major causes of hormone refractory prostate cancer. Moreover, the malfunction of AR has been implicated in several types of disease. Without wishing to be bound by any theory, it is believed that an AR inhibitor that acts as an inverse agonist to AR can be a viable pathway to treating disorders associated with AR overexpression and/or malfunction. Accordingly, the present invention provides a method for treating a disorder associated with androgen receptor malfunction in a subject in need thereof comprising administering to the subject an effective amount of the compound of formula (I), including formula (Ia), or a pharmaceutically acceptable salt thereof to the subject. A disorder associated with androgen receptor malfunction can be, for example, a hyperproliferative disorder, such as a hormone-sensitive cancer, a castrate-sensitive cancer, a metastatic castrate-sensitive cancer, a hormone refractory cancer, castrate-resistant cancer, and a metastatic castrate-resistant cancer. In some instances, the disorder associated with androgen receptor malfunction is prostate cancer, which can be hormone-sensitive prostate cancer or hormone refractory prostate cancer.

In certain aspects of this method, the compound of formula (I), including a compound of formula (Ia), or a pharmaceutically acceptable salt thereof can be co-administered with an additional anti-cancer therapy, including androgen-deprivation therapy (ADT), including orchiectomy and/or anti-androgen therapy (e.g., the administration of at least one androgen-suppressing agent), chemotherapy (e.g., the administration of at least one chemotherapeutic agent), and/or radiation therapy. In an aspect, the method comprises administering an amount of a compound of formula (I) or a salt thereof that is effective to sensitize the cancer cells to one or more therapeutic regimens (e.g., ADT, chemotherapy, or radiation therapy). The terms “co-administered” or “co-administration” refer to simultaneous or sequential administration. A compound may be administered before, concurrently with, or after administration of another therapy.

Agents used for suppressing either the production or activity of androgens, include a luteinizing hormone-releasing hormone (LHRH) agonist (e.g., leuprolide, goserelin, triptorelin, histrelin), an LHRH antagonist (e.g., degarelix, relugolix), abiraterone, ketoconazole, enzalutamide, apalutamide, darolutamide, flutamide, bicalutamide, nilutamide, estrogens, or any combination thereof.

In the case of chemotherapy, one or more than one, e.g., two, three, or more anti-cancer agents can be administered. In this regard, the present invention is directed a pharmaceutical composition comprising a pharmaceutically acceptable carrier and a combination of the compound of formula (I), including a compound of formula (Ia), or a pharmaceutically acceptable salt thereof and at least one anti-cancer agent (e.g., chemotherapeutic agent).

Examples of anti-cancer agents include platinum compounds (e.g., cisplatin, carboplatin, oxaliplatin), alkylating agents (e.g., cyclophosphamide, ifosfamide, chlorambucil, nitrogen mustard, thiotepa, melphalan, busulfan, procarbazine, streptozocin, temozolomide, dacarbazine, bendamustine), antitumor antibiotics (e.g., daunorubicin, doxorubicin, idarubicin, epirubicin, mitoxantrone, bleomycin, mytomycin C, plicamycin, dactinomycin), taxanes (e.g., paclitaxel and docetaxel), antimetabolites (e.g., 5-fluorouracil, cytarabine, premetrexed, thioguanine, floxuridine, capecitabine, and methotrexate), nucleoside analogues (e.g., fludarabine, clofarabine, cladribine, pentostatin, nelarabine), topoisomerase inhibitors (e.g., topotecan and irinotecan), hypomethylating agents (e.g., azacitidine and decitabine), proteosome inhibitors (e.g., bortezomib), epipodophyllotoxins (e.g., etoposide and teniposide), DNA synthesis inhibitors (e.g., hydroxyurea), vinca alkaloids (e.g., vicristine, vindesine, vinorelbine, and vinblastine), tyrosine kinase inhibitors (e.g., imatinib, dasatinib, nilotinib, sorafenib, sunitinib), monoclonal antibodies (e.g., rituximab, cetuximab, panetumumab, tositumomab, trastuzumab, alemtuzumab, gemtuzumab ozogamicin, bevacizumab), nitrosoureas (e.g., carmustine, fotemustine, and lomustine), enzymes (e.g., L-Asparaginase), biological agents (e.g., interferons and interleukins), hexamethylmelamine, mitotane, angiogenesis inhibitors (e.g., thalidomide, lenalidomide), steroids (e.g., abiraterone acetate, prednisone, dexamethasone, and prednisolone), hormonal agents (e.g., tamoxifen, raloxifene, leuprolide, bicalutamide, granisetron, flutamide), aromatase inhibitors (e.g., letrozole and anastrozole), arsenic trioxide, tretinoin, nonselective cyclooxygenase inhibitors (e.g., nonsteroidal anti-inflammatory agents, salicylates, aspirin, piroxicam, ibuprofen, indomethacin, naprosyn, diclofenac, tolmetin, ketoprofen, nabumetone, oxaprozin), selective cyclooxygenase-2 (COX-2) inhibitors, or any combination thereof.

For purposes of the present invention, the term “subject” preferably is directed to a mammal. Mammals include, but are not limited to, the order Rodentia, such as mice, and the order Lagomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perissodactyla, including Equines (horses). It is most preferred that the mammals are of the order Primates, Cebids, or Simioids (monkeys) or of the order Anthropoids (humans and apes). An especially preferred mammal is a human.

The invention is further illustrated by the following aspects.

(1) A compound of formula (I)

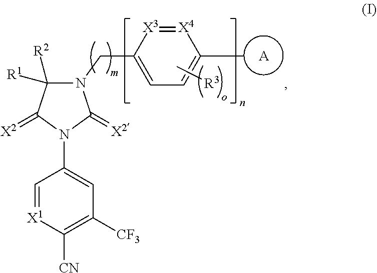

wherein R¹ and R² are the same or different and each is alkyl, X¹ is CH or N, X² and X² are the same or different and each is O or S, X³ and X⁴ are the same or different and each is CR³ or N, R³ is hydrogen, halo, hydroxy, alkyl, or alkoxy, ring A is selected from the group consisting of

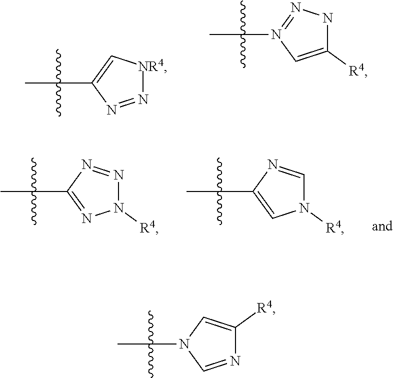

R⁴ is hydrogen, alkyl, cycloalkyl, heterocycloalkyl, aryl, or heteroaryl, wherein the alkyl, cycloalkyl, heterocycloalkyl, aryl, or heteroaryl is optionally substituted, m is 0-8, n is 0 or 1, such that 1≤m+n≤9, and o is 0-2, provided that when X¹ is CH, X³ and X⁴ are each CR³, ring A is

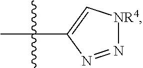

R⁴ is hydrogen or alkyl, n is 1, and o is 0-2, then at least one R³ is not hydrogen, or a pharmaceutically acceptable salt thereof.

(2) The compound of aspect 1, wherein R¹ and R² are each methyl, or a pharmaceutically acceptable salt thereof.

(3) The compound of aspect 1 or 2, wherein X¹ is N, or a pharmaceutically acceptable salt thereof.

(4) The compound of aspect 1 or 2, wherein X¹ is CH, or a pharmaceutically acceptable salt thereof.

(5) The compound of any one of aspects 1-4, wherein X³ and X⁴ are each CR³, or a pharmaceutically acceptable salt thereof.

(6) The compound of aspect 1, wherein the compound of formula (I) is a compound of formula (Ia)

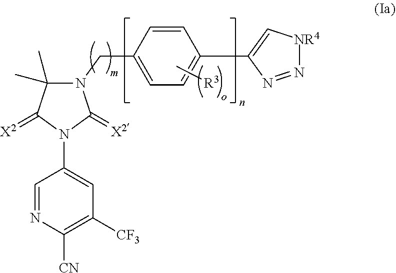

wherein X² and X² are the same or different and each is O or S, R³ is hydrogen, halo, hydroxy, alkyl, or alkoxy, R⁴ is hydrogen, alkyl, cycloalkyl, heterocycloalkyl, aryl, or heteroaryl, wherein the alkyl, cycloalkyl, heterocycloalkyl, aryl, or heteroaryl is optionally substituted, m is 0-8, n is 0 or 1, such that 1≤m+n≤9, and o is 0-2, or a pharmaceutically acceptable salt thereof.

(7) The compound of any one of aspects 1-6, wherein at least one R³ moiety is fluoro, or a pharmaceutically acceptable salt thereof.

(8) The compound of any one of aspects 1-7, wherein X² is O and X^{2′} is S, or a pharmaceutically acceptable salt thereof.

(9) The compound of any one of aspects 1-7, wherein X² and X² are O, or a pharmaceutically acceptable salt thereof.

(10) The compound of any one of aspects 1-9, wherein m is 1, or a pharmaceutically acceptable salt thereof.

(11) The compound of any one of aspects 1-10, wherein n is 1, or a pharmaceutically acceptable salt thereof.

(12) The compound of any one of aspects 1-11, wherein R⁴ is hydrogen, or a pharmaceutically acceptable salt thereof.

(13) The compound of any one of aspects 1-11, wherein R⁴ is alkyl that is optionally substituted, or a pharmaceutically acceptable salt thereof.

(14) The compound of aspect 13, wherein the alkyl is substituted with at least one substituent selected from hydroxyl, cyano, amino, alkylamino, dialkylamino, halo, nitro, carboxy, amido, cycloalkyl, heterocycloalkyl, aryl, and heteroaryl, or a pharmaceutically acceptable salt thereof.

(15) The compound of aspect 1 that is, wherein R³ is not hydrogen:

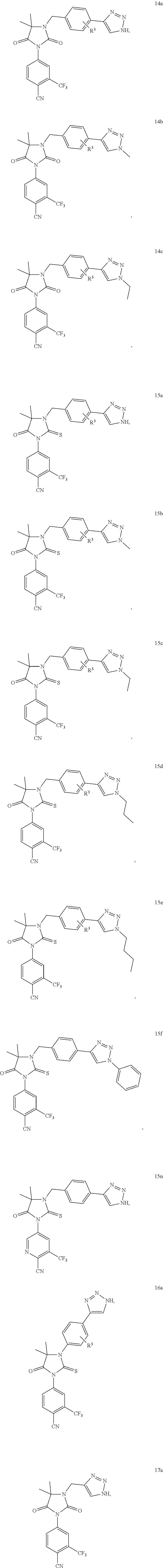

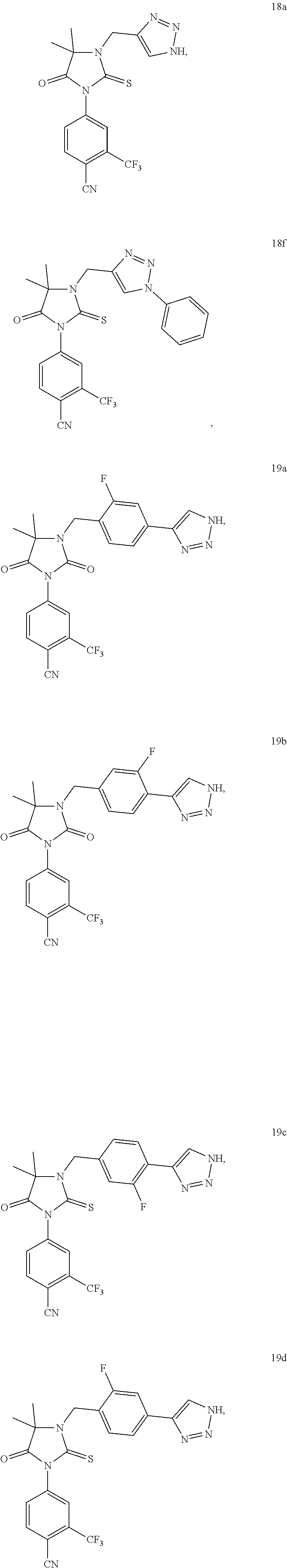 [SMILES: CC1(C)C(=O)N(c2ccc(C#N)c(C(F)(F)F)c2)C(=S)N1Cc1c[nH]nn1 ; CC1(C)C(=O)N(c2ccc(C#N)c(C(F)(F)F)c2)C(=S)N1Cc1cn(-c2ccccc2)nn1 ; CC1(C)C(=O)N(c2ccc(C#N)c(C(F)(F)F)c2)C(=O)N1Cc1ccc(-c2c[nH]nn2)cc1F ; CC1(C)C(=O)N(c2ccc(C#N)c(C(F)(F)F)c2)C(=O)N1Cc1ccc(-c2c[nH]nn2)c(F)c1 ; CC1(C)C(=O)N(c2ccc(C#N)c(C(F)(F)F)c2)C(=S)N1Cc1ccc(-c2c[nH]nn2)c(F)c1 ; CC1(C)C(=O)N(c2ccc(C#N)c(C(F)(F)F)c2)C(=S)N1Cc1ccc(-c2c[nH]nn2)cc1F]

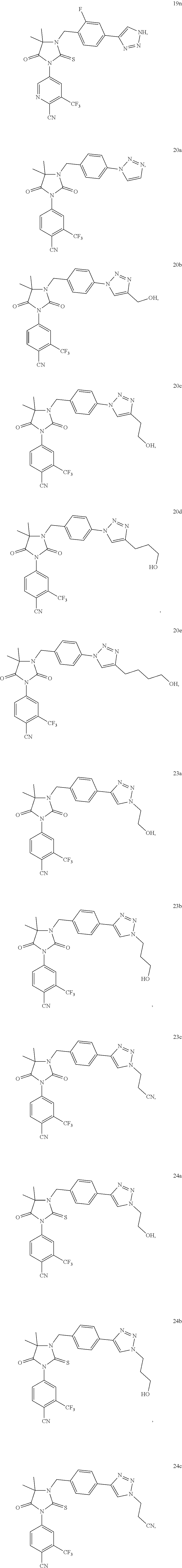

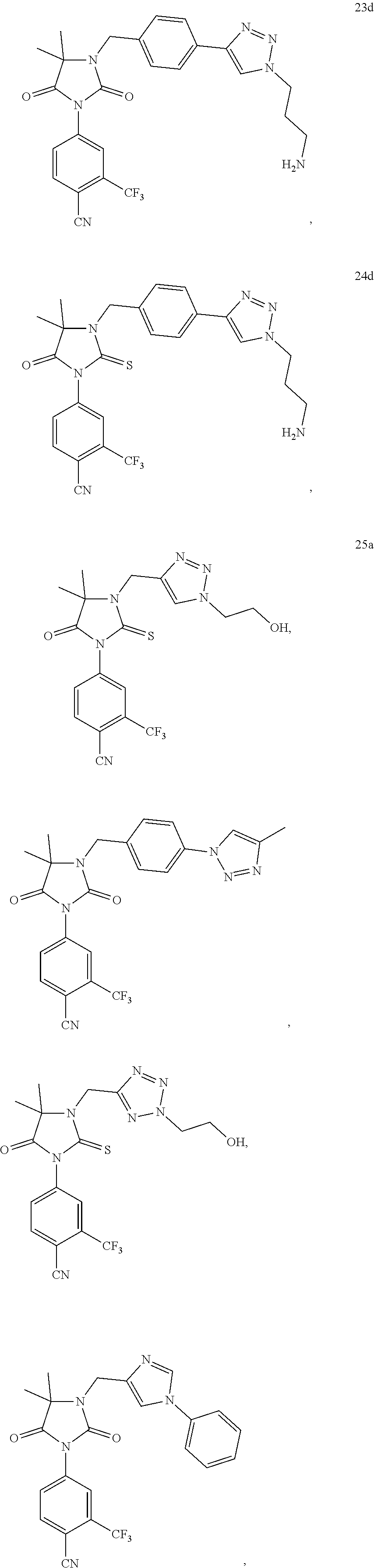 [SMILES: CC1(C)C(=O)N(c2ccc(C#N)c(C(F)(F)F)c2)C(=O)N1Cc1ccc(-c2cn(CCCN)nn2)cc1 ; CC1(C)C(=O)N(c2ccc(C#N)c(C(F)(F)F)c2)C(=S)N1Cc1ccc(-c2cn(CCCN)nn2)cc1 ; CC1(C)C(=O)N(c2ccc(C#N)c(C(F)(F)F)c2)C(=S)N1Cc1cn(CCO)nn1 ; Cc1cn(-c2ccc(CN3C(=O)N(c4ccc(C#N)c(C(F)(F)F)c4)C(=O)C3(C)C)cc2)nn1 ; CC1(C)C(=O)N(c2ccc(C#N)c(C(F)(F)F)c2)C(=S)N1Cc1nnn(CCO)n1 ; CC1(C)C(=O)N(c2ccc(C#N)c(C(F)(F)F)c2)C(=O)N1Cc1cn(-c2ccccc2)cn1]

[chemical structure]

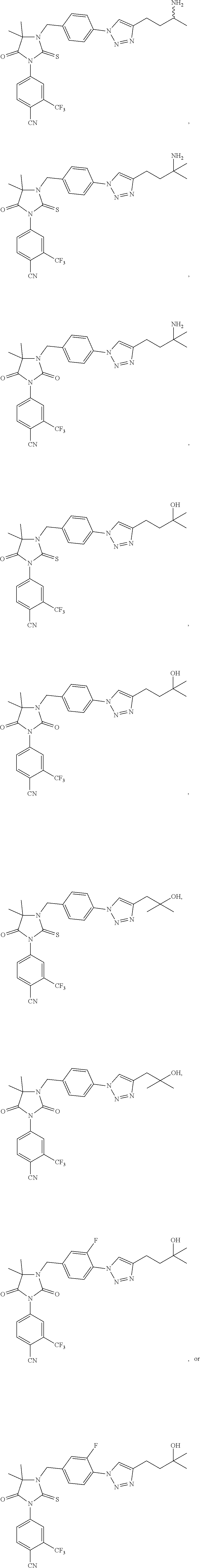

or a pharmaceutically acceptable salt thereof.

(16) The compound of aspect 14 that is

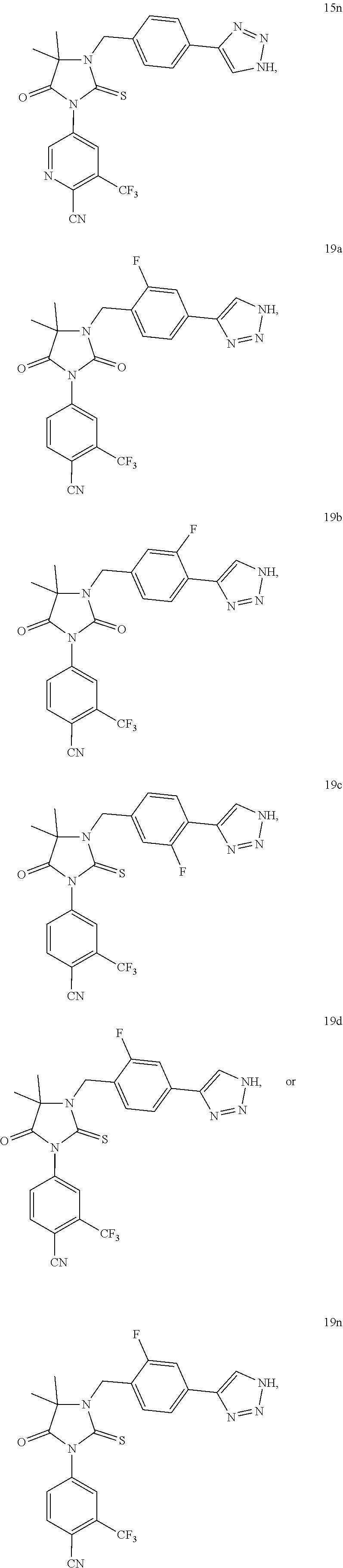 [SMILES: CC1(C)C(=O)N(c2cnc(C#N)c(C(F)(F)F)c2)C(=S)N1Cc1ccc(-c2c[nH]nn2)cc1 ; CC1(C)C(=O)N(c2ccc(C#N)c(C(F)(F)F)c2)C(=O)N1Cc1ccc(-c2c[nH]nn2)cc1F ; CC1(C)C(=O)N(c2ccc(C#N)c(C(F)(F)F)c2)C(=O)N1Cc1ccc(-c2c[nH]nn2)c(F)c1 ; CC1(C)C(=O)N(c2ccc(C#N)c(C(F)(F)F)c2)C(=S)N1Cc1ccc(-c2c[nH]nn2)c(F)c1 ; CC1(C)C(=O)N(c2ccc(C#N)c(C(F)(F)F)c2)C(=S)N1Cc1ccc(-c2c[nH]nn2)cc1F ; CC1(C)C(=O)N(c2ccc(C#N)c(C(F)(F)F)c2)C(=S)N1Cc1ccc(-c2c[nH]nn2)cc1F]

or a pharmaceutically acceptable salt thereof.

(17) A pharmaceutical composition comprising the compound of any one of aspects 1-16, or a pharmaceutically acceptable salt thereof, and a pharmaceutically acceptable carrier.

(18) A method of treating a disorder associated with androgen receptor malfunction in a subject in need thereof comprising administering to the subject an effective amount of the compound of any one of aspects 1-16 or a pharmaceutically acceptable salt thereof.

(19) The method of aspect 18, wherein the disorder associated with androgen receptor malfunction is a hyperproliferative disorder.

(20) The method of aspect 18 or 19, wherein the disorder associated with androgen receptor malfunction is a hormone-sensitive cancer.

(21) The method of aspect 18 or 19, wherein the disorder associated with androgen receptor malfunction is a hormone refractory cancer.

(22) The method of any one of aspects 18-21, wherein the disorder associated with androgen receptor malfunction is prostate cancer.

The following examples further illustrate the invention but, of course, should not be construed as in any way limiting its scope.

### EXAMPLES

Enzalutamide was purchased from Selleckchem (Houston, Tex.). Bicalutamide and testosterone were a kind gift from Dr. Shafiq Khan (Clark Atlanta University, Atlanta, Ga.). All other chemicals (including SAHA) were purchased from Sigma Aldrich (St. Louis, Mo.). Anhydrous solvents and other reagents were purchased and used without further purification. Analtech silica gel plates (60 F₂₅₄) were used for analytical TLC, and Analtech preparative TLC plates (UV 254, 2000 μm) were used for purification. UV light was used to examine the spots. 200-400 Mesh silica gel was used in column chromatography. Nuclear magnetic resonance (NMR) spectra were recorded on a Varian-Gemini 400 magnetic resonance spectrometer. ¹H NMR spectra are recorded in parts per million (ppm) relative to the peak of CDCl₃, (7.24 ppm), CD₃OD (3.31 ppm), or DMSO-d₆ (2.49 ppm). ¹³C spectra were recorded relative to the central peak of the CDCl₃ triplet (77.16 ppm), CD₃OD (49.0 ppm), or the DMSO-d₆ septet (39.7 ppm), and were recorded with complete hetero-decoupling. Multiplicities are described using the abbreviation s, singlet; d, doublet; t, triplet; q, quartet; quin, quintet; sex, sextet; m, multiplet. All biologically evaluated compounds were established to be >95% pure using high-performance liquid chromatography (HPLC). High-resolution mass spectra were recorded at the Georgia Institute of Technology mass spectrometry facility in Atlanta. Common abbreviations include: TBTU (O-(benzotriazol-1-yl)-N,N,N′,N′-tetramethyluronium tetrafluoroborate), DMF (N,N′-dimethylformamide), DCM (dichloromethane), TLC (thin layer chromatography), THF (tetrahydrofuran), DIPEA (N,N′-diisopropylethylamine), and DMSO (dimethyl sulfoxide).

### Example 1

This example demonstrates a synthesis of cyano-nilutamide (4).

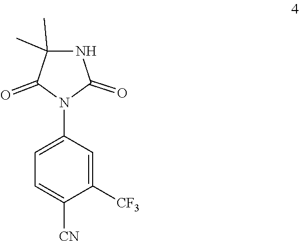 [SMILES: CC1(C)NC(=O)N(c2ccc(C#N)c(C(F)(F)F)c2)C1=O]

4-Fluoro-2-(trifluoromethyl)benzonitrile (4.02 g, 21.3 mmol) was added to hydantoin (13.6 g, 106.3 mmol) and potassium carbonate (4.40 g, 31.9 mmol) in 60 mL DMF and stirred at 45° C. under argon for 48 hours. The reaction mixture was then diluted in ethyl acetate and washed three times with water. The organic layer was dried over sodium sulfate, filtered, and concentrated in vacuo. Column chromatography (eluent 30:1 DCM/methanol) gave 4 as a white solid (4.62 g, 74%). ¹H NMR (400 MHz, (CD₃)₂-CO) δ 1.54 (6H, s), 7.80 (1H, s), 8.13 (1H, dd, J=1.8 Hz, J=8.4 Hz), 8.20 (1H, d, J=8.4 Hz), 8.26 (1H, d, J=1.8 Hz).

### Example 2

This example demonstrates a synthesis of cyano-nilutamide-benzyl-alkyne (i.e., 4-[3-[(4-ethynylphenyl)methyl]-4,4-dimethyl-2,5-dioxo-1-imidazolidinyl]-2-(trifluoromethyl)-benzonitrile) (6).

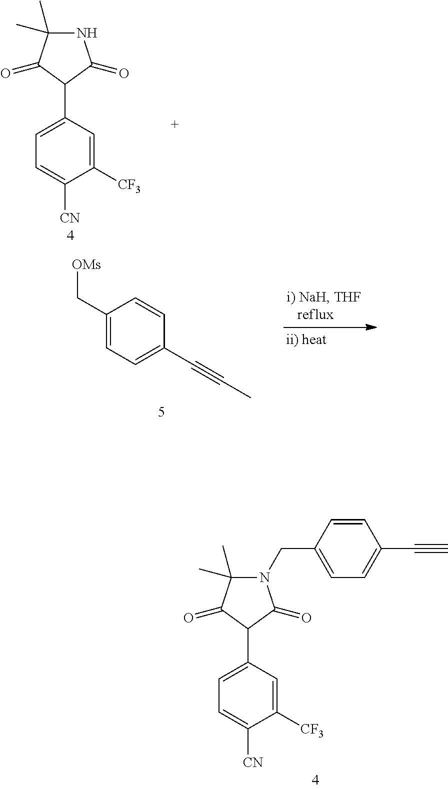 [SMILES: CC1(C)NC(=O)C(c2ccc(C#N)c(C(F)(F)F)c2)C1=O ; "+" ; CC#Cc1ccc(COS(C)(=O)=O)cc1 ; C#Cc1ccc(CN2C(=O)C(c3ccc(C#N)c(C(F)(F)F)c3)C(=O)C2(C)C)cc1]

Compound 4 (2.44 g, 8.20 mmol) was dissolved in 28 mL DMF under argon, followed by addition of NaH (60% in mineral oil, 558 mg, 13.9 mmol) and stirring for 2 hours at ambient temperature. Then 5 (3.27 g, 15.5 mmol) was added, and the reaction was stirred for 5 hours at 53° C. The mixture was then dissolved in 150 mL EtOAc and washed 5 times with 125 mL brine, and 3 times with 125 mL H₂O. The organic layer was dried over sodium sulfate, filtered, and concentrated in vacuo. Trituration with MeOH:H₂O (7:1) gave 6 as a white solid (quantitative yield). ¹H NMR (400 MHz, CDCl₃) (1.37 (6H, s), 3.09 (1H, s), 4.57 (2H, s), 7.30 (2H, d, J=8.4 Hz), 7.41 (2H, d, J=8.3 Hz), 7.86 (1H, d, J=8.4 Hz), 8.00 (1H, dd, J=1.9, 8.4 Hz), 8.14 (1H, d, J=7.3 Hz) ppm.

### Example 3

This example demonstrates a synthesis of methyl 2-((4-ethynylbenzyl)amino)-2-methylpropanoate (7).

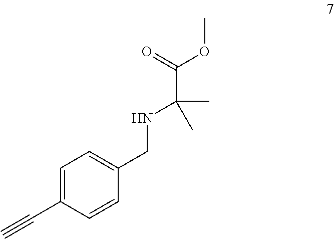 [SMILES: C#Cc1ccc(CNC(C)(C)C(=O)OC)cc1]

Methyl 2-amino-2-methylpropanoate hydrochloride (1.70 g, 11.1 mmol), K₂CO₃ (10.7 g, 77.6 mmol) and 5 (2.80 g, 13.3 mmol) were dissolved in acetonitrile under argon and set to stir. The reaction was heated to 77° C. and run overnight. The mixture was then diluted with DCM and washed 5 times with 0.5 N HCl. The aqueous layers were combined and basified with NaOH pellets to achieve pH=12, followed by extraction with DCM:MeOH:NH₄OH (10:1:0.1). The organic layer was dried with Na₂SO₄ and concentrated in vacuo to obtain 1.36 g (53% yield) of 7, which was used without further purification. ¹H NMR (400 MHz, CDCl₃) δ 1.35 (6H, s), 1.84 (1H, s), 3.04 (1H, s), 3.62 (2H, s), 3.72 (3H, s), 7.29 (2H, d, J=8.5 Hz), 7.43 (2H, d, J=8.2 Hz) ppm. ¹³C NMR (400 MHz, CDCl₃) δ 177.38, 141.31, 132.17, 129.58, 128.18, 120.67, 83.61, 76.89, 59.32, 51.96, 48.66, 25.42.

### Example 4

This example demonstrates a synthesis of 4-isothiocyanato-2-(trifluoromethyl)benzonitrile (9).

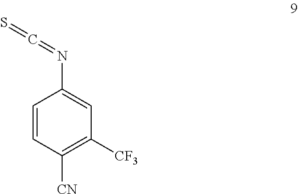 [SMILES: N#Cc1ccc(N=C=S)cc1C(F)(F)F]

4-Amino-2-(trifluoromethyl)benzonitrile (4.46 g, 24.0 mmol) and 2.2 mL thiophosgene were reacted in water (44 mL) for 1 hour at room temperature. The reaction mixture was extracted with chloroform, dried with Na₂SO₄, and concentrated in vacuo to obtain 9 as a dark orange solid (5.13 g, 94% yield), which was used without further purification. ¹H NMR (400 MHz, CDCl₃) δ 7.50 (1H, dd, J=2.0, 8.3 Hz), 7.56 (1H, d, J=2.0 Hz), 7.84 (1H, d, J=8.3 Hz) ppm.

### Example 5

This example demonstrates a synthesis of thiohydantoin benzyl alkyne (i.e., 4-(3-(4-ethynylbenzyl)-4,4-dimethyl-5-oxo-2-thioxoimidazolidin-1-yl)-2-(trifluoromethyl)benzonitrile) (10).

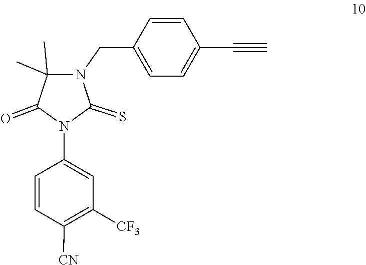 [SMILES: C#Cc1ccc(CN2C(=S)N(c3ccc(C#N)c(C(F)(F)F)c3)C(=O)C2(C)C)cc1]

Isothiocyanate 9 (1.02 g, 4.47 mmol) and 7 (940 mg, 4.07 mmol) were dissolved in THF under argon and set to stir at 50° C. for 3 hours. 70 mg of 9 was added, and heated to reflux, to push reaction to completion. THF was removed under reduced pressure, the crude product was redissolved in 0.5N HCl, extracted 3 times with DCM, dried with Na₂SO₄, and concentrated in vacuo to obtain 1.71 g (99% yield) of 10 as a white solid, which was used without further purification. ¹H NMR (400 MHz, cdcl3) δ 1.45 (6H, s), 3.11 (1H, s), 5.11 (2H, s), 7.38 (2H, d, J=8.5 Hz), 7.48 (2H, d, J=8.4 Hz), 7.82 (1H, dd, J=2.0, 8.3 Hz), 7.94 (1H, d, J=2.1 Hz), 7.97 (1H, d, J=8.3 Hz) ppm. ¹³C NMR (101 MHz, cdcl3) δ 180.09, 175.34, 137.35, 136.94, 135.29, 132.71, 132.26, 127.76, 127.17, 127.12, 123.34, 122.20, 120.61, 114.93, 110.24, 83.05, 78.14, 65.45, 47.29, 23.73.

### Example 6

This example demonstrates a synthesis of thiohydantoin phenyl alkyne (i.e., 4-(3-(4-ethynylphenyl)-4,4-dimethyl-5-oxo-2-thioxoimidazolidin-1-yl)-2-(trifluoromethyl)benzonitrile) (13).

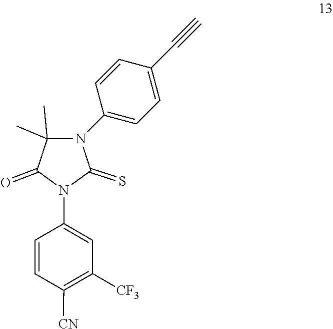 [SMILES: C#Cc1ccc(N2C(=S)N(c3ccc(C#N)c(C(F)(F)F)c3)C(=O)C2(C)C)cc1]

Step 1. Ethyl 2-((4-bromophenyl)amino)-2-methylpropanoate (2.00 g, 7.00 mmol) was added to CuI (133 mg, 0.70 mmol), Pd(Cl₂)(PPh₃)₂ (491 mg, 0.70 mmol), and ethynyltrimethylsilane (825 mg, 8.40 mmol) in TEA (25 mL) under argon. The reaction was set to stir and heated to 77° C. for 18 hours. TEA was evaporated, the crude mixture was dissolved in ethyl acetate, extracted with saturated NH₄Cl and concentrated. Column chromatography (eluent 40:1:1, hexane:DCM:ethyl acetate) provided 1.77 g (83.3% yield) of ethyl 2-methyl-2-((4-((trimethylsilyl)ethynyl)phenyl)amino)propanoate. ¹H NMR (400 MHz, cdcl3) δ 0.21 (9H, s), 1.15 (3H, t, J=7.1 Hz), 1.54 (6H, s), 4.13 (3H, q, J=7.1 Hz), 6.43 (2H, d, J=8.8 Hz), 7.24 (2H, d, J=8.8 Hz) ppm. ¹³C NMR (101 MHz, cdcl3) δ 175.80, 145.97, 133.15, 114.23, 111.97, 106.15, 91.58, 61.50, 57.33, 26.24, 14.20, 0.25.

Step 2. Isothiocyanate 9 (1.33 g, 5.83 mmol) and ethyl 2-methyl-2-((4-((trimethylsilyl)ethynyl)phenyl)amino)propanoate (1.47 g, 4.86 mmol) were dissolved in THF (40 mL) and set to stir at 55° C. for 13 hours. Next, 232 mg 9 and 10 mL DMSO were then added, and the temperature was increased to 87° C. for 24 hours. The reaction was cooled, taken up in ethyl acetate, washed 4 times with H₂O, dried over Na₂SO₄, and concentrated in vacuo. Column chromatography (gradient eluent, hexane:ethyl acetate, from 80:1 to 7:1) afforded 1.15 g (48% yield) of the TMS-alkyne. This was then reacted with K₂CO₃ (1.63 g, 11.8 mmol) in methanol for 1 hour, diluted with diethyl ether, washed 3 times with water, dried over Na₂SO₄ and concentrated in vacuo to obtain 13 (976 mg, 100% yield) as an off-white solid. ¹H NMR (400 MHz, cdcl3) δ 1.59 (6H, s), 3.20 (1H, s), 7.28 (2H, d, J=8.2 Hz), 7.65 (2H, d, J=8.3 Hz), 7.84 (1H, dd, J=1.8, 8.2 Hz), 7.95-8.01 (2H, m) ppm. ¹³C NMR (101 MHz, cdcl3) δ 179.75, 174.79, 137.03, 135.24, 133.58, 132.20, 129.63, 127.12, 127.07, 123.97, 114.80, 110.18, 82.15, 79.51, 66.51, 23.74.

### Example 7

This example demonstrates a synthesis of 4-(3-(4-(1H-1,2,3-triazol-4-yl)benzyl)-4,4-dimethyl-2,5-dioxoimidazolidin-1-yl)-2-(trifluoromethyl)benzonitrile (14a), which is representative for arylhydantoin triazoles, such as 14a, 15a, 16a, 17a, and 18a.

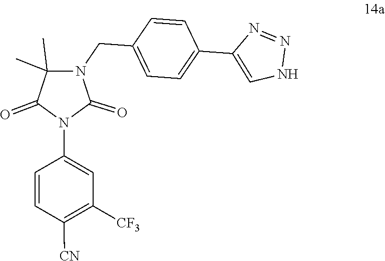 [SMILES: CC1(C)C(=O)N(c2ccc(C#N)c(C(F)(F)F)c2)C(=O)N1Cc1ccc(-c2c[nH]nn2)cc1]

Hydantoin benzyl alkyne 6 (81.3 mg, 0.198 mmol) was dissolved in DMF (1.6 mL) and MeOH (0.4 mL) under argon. TMS-N₃ (34.2 mg, 0.296 mmol) and CuI (3.8 mg, 0.020 mmol) were then added, and the reaction was heated at 100° C. overnight. The mixture was evaporated, dissolved in ethyl acetate, washed 4 times with 30 mL portions of NH₄Cl:NH₄OH (4:1), dried over Na₂SO₄, and concentrated in vacuo to obtain 14a (25.7 mg, 28.6% yield) as an off-white solid. ¹H NMR (400 MHz, cdcl3) δ 1.45 (6H, s), 4.67 (2H, s), 7.46 (2H, d, J=8.1 Hz), 7.82 (2H, d, J=7.9 Hz), 7.93 (1H, d, J=8.5 Hz), 8.00 (1H, s), 8.04 (1H, dd, J=2.0, 8.5 Hz), 8.19 (1H, d, J=1.7 Hz) ppm. ¹³C NMR (101 MHz, cdcl3) δ 174.56, 153.37, 137.34, 136.37, 135.32, 133.82, 133.49, 129.90, 128.65, 128.04, 126.63, 123.28, 123.18, 123.13, 123.08, 123.03, 120.56, 114.98, 108.38, 62.28, 43.50, 23.71.

### Example 8

This example demonstrates a synthesis of 4-(3-(4-(1H-1,2,3-triazol-4-yl)benzyl)-4,4-dimethyl-5-oxo-2-thioxoimidazolidin-1-yl)-2-(trifluoromethyl)benzonitrile (15a).

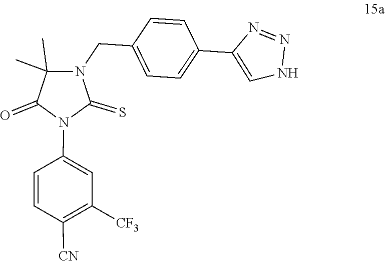 [SMILES: CC1(C)C(=O)N(c2ccc(C#N)c(C(F)(F)F)c2)C(=S)N1Cc1ccc(-c2c[nH]nn2)cc1]

Thiohydantoin benzyl alkyne 10 (120 mg, 0.281 mmol) was reacted under the same conditions and purified just as 14a to obtain 15a (60 mg, 44.1% yield) as an orange solid. ¹H NMR (400 MHz, cdcl3) δ 1.46 (6H, s), 5.16 (2H, s), 7.51 (2H, d, J=8.1 Hz), 7.77-7.86 (3H, m), 7.92-7.96 (1H, m), 7.98 (1H, s), 7.98 (1H, s) ppm. ¹³C NMR (101 MHz, cdcl3) δ 180.06, 175.49, 137.40, 136.68, 135.32, 133.78, 133.45, 132.30, 130.08, 128.46, 127.21, 127.16, 126.70, 123.34, 120.61, 114.94, 110.19, 65.53, 47.35, 23.76.

### Example 9

This example demonstrates a synthesis of 4-(3-(4-(1H-1,2,3-triazol-4-yl)phenyl)-4,4-dimethyl-5-oxo-2-thioxoimidazolidin-1-yl)-2-(trifluoromethyl)benzonitrile (16a).

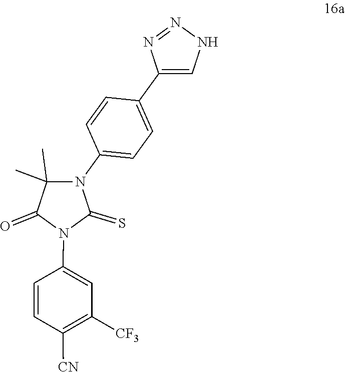 [SMILES: CC1(C)C(=O)N(c2ccc(C#N)c(C(F)(F)F)c2)C(=S)N1c1ccc(-c2c[nH]nn2)cc1]

Thiohydantoin phenyl alkyne 13 (150 mg, 0.364 mmol) was reacted under the same conditions as 14a. The mixture was evaporated, dissolved in ethyl acetate, washed 4 times with 30 mL portions of saturated NH₄Cl, dried over Na₂SO₄, and concentrated in vacuo. Preparative TLC, mobile phase 30:1 DCM:MeOH, yielded 16a (81.6 mg, 49.1% yield) as an off-white solid. ¹H NMR (400 MHz, cdcl3) δ 1.62 (7H, s), 7.39 (2H, d, J=8.2 Hz), 7.86 (1H, dd, J=1.6, 8.3 Hz), 7.95-8.02 (4H, m), 8.04 (1H, s) ppm. ¹³C NMR (101 MHz, cdcl3) δ 179.99, 175.08, 137.14, 135.39, 135.10, 133.74, 133.41, 132.35, 131.61, 130.25, 127.55, 127.24, 127.19, 123.28, 120.56, 114.90, 110.19, 66.70, 23.79.

### Example 10

This example demonstrates a synthesis for 4-(4,4-dimethyl-3-(4-(1-methyl-1H-1,2,3-triazol-4-yl)benzyl)-2,5-dioxoimidazolidin-1-yl)-2-(trifluoromethyl)benzonitrile (14b), which is representative for alkylated arylhydantoin triazoles (14b and 15b-e):

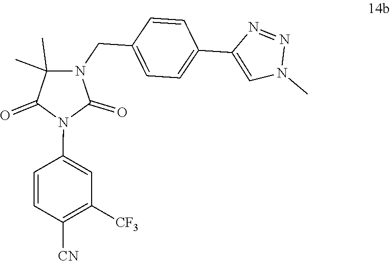 [SMILES: Cn1cc(-c2ccc(CN3C(=O)N(c4ccc(C#N)c(C(F)(F)F)c4)C(=O)C3(C)C)cc2)nn1]

Hydantoin benzyl alkyne 6 (74.5 mg, 0.181 mmol) was dissolved in ethanol (3 mL), to which was added a mixture of NaN₃ (17.7 mg, 0.272 mmol) and CH₃I (17 μL, 0.272 mmol) in water (0.2 mL) and ethanol (0.2 mL), then CuI (3.4 mg, 0.018 mmol) and DIPEA (47.3 μL, 0.272 mmol) were added, and set to stir overnight at room temperature. The reaction was quenched with 40 mL water and extracted three times with DCM (20 mL), concentrated, and purified with preparative TLC (eluent, 30:1 DCM:MeOH) to obtain 14b as a white solid (32.4 mg, 38.2% yield). ¹H NMR (400 MHz, cdcl3) 61.43 (6H, s), 4.14 (3H, s), 4.64 (2H, s), 7.42 (2H, d, J=8.1 Hz), 7.76 (1H, s), 7.80 (2H, d, J=8.1 Hz), 7.92 (1H, d, J=8.5 Hz), 8.04 (1H, dd, J=2.1, 8.4 Hz), 8.19 (1H, d, J=1.8 Hz) ppm. ¹³C NMR (101 MHz, cdcl3) δ 174.57, 153.27, 136.72, 136.46, 135.27, 133.73, 133.40, 130.55, 128.57, 127.98, 126.16, 123.31, 123.05, 123.00, 120.77, 115.01, 108.27, 62.23, 43.48, 36.81, 23.68.

### Example 11

This example demonstrates a synthesis of 4-(4,4-dimethyl-3-(4-(1-methyl-1H-1,2,3-triazol-4-yl)benzyl)-5-oxo-2-thioxoimidazolidin-1-yl)-2-(trifluoromethyl)benzonitrile (15b).

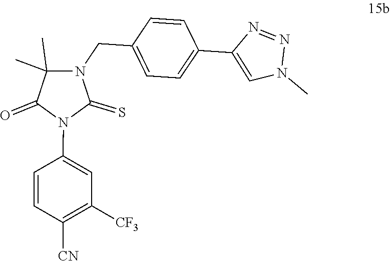 [SMILES: Cn1cc(-c2ccc(CN3C(=S)N(c4ccc(C#N)c(C(F)(F)F)c4)C(=O)C3(C)C)cc2)nn1]

Thiohydantoin benzyl alkyne 10 (87.1 mg, 0.204 mmol) was reacted under the same conditions and purified just as 14b to obtain 15b (50.3 mg, 51.0% yield) as an off-white solid. ¹H NMR (400 MHz, cdcl3) δ 1.46 (7H, s), 4.13 (3H, s), 5.14 (2H, s), 7.47 (2H, d, J=7.9 Hz), 7.73-7.87 (4H, m), 7.93-8.00 (2H, m) ppm. ¹³C NMR (101 MHz, cdcl3) δ 179.95, 175.42, 137.41, 136.05, 135.26, 133.67, 133.34, 132.29, 130.69, 128.36, 127.12, 126.22, 123.33, 120.93, 120.60, 114.94, 110.09, 65.48, 47.36, 36.93, 23.73.

### Example 12

This example demonstrates a synthesis of 4-(3-(4-(1-ethyl-1H-1,2,3-triazol-4-yl)benzyl)-4,4-dimethyl-5-oxo-2-thioxoimidazolidin-1-yl)-2-(trifluoromethyl)benzonitrile (15c).

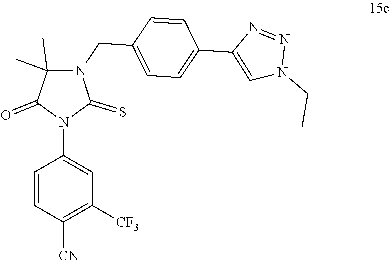 [SMILES: CCn1cc(-c2ccc(CN3C(=S)N(c4ccc(C#N)c(C(F)(F)F)c4)C(=O)C3(C)C)cc2)nn1]

Thiohydantoin benzyl alkyne 10 (48 mg, 0.112 mmol) and EtI (14 μL, 0.176 mmol) were reacted under the same conditions and purified just as 14b to obtain 15c (26.8 mg, 47.9% yield) as an off-white solid. ¹H NMR (400 MHz, cdcl3) δ 1.47 (6H, s), 1.61 (3H, t, J=7.0 Hz), 4.47 (2H, q, J=7.1 Hz), 5.15 (2H, s), 7.48 (2H, d, J=7.6 Hz), 7.79 (1H, s), 7.81-7.87 (3H, m), 7.92-8.02 (2H, m) ppm. ¹³C NMR (101 MHz, cdcl3) δ 180.01, 175.48, 137.43, 136.03, 135.29, 133.80, 133.47, 132.30, 130.95, 128.43, 127.22, 127.17, 126.28, 119.18, 114.95, 110.19, 65.51, 47.46, 45.60, 23.80, 15.72.

### Example 13

This example demonstrates a synthesis of 4-(4,4-dimethyl-5-oxo-3-(4-(1-propyl-1H-1,2,3-triazol-4-yl)benzyl)-2-thioxoimidazolidin-1-yl)-2-(trifluoromethyl)benzonitrile (15d).

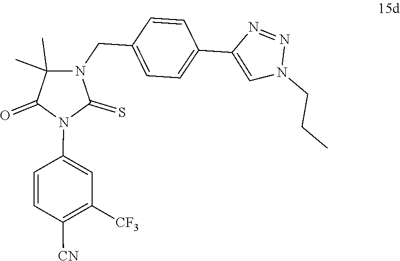 [SMILES: CCCn1cc(-c2ccc(CN3C(=S)N(c4ccc(C#N)c(C(F)(F)F)c4)C(=O)C3(C)C)cc2)nn1]

Thiohydantoin benzyl alkyne 10 (42 mg, 0.098 mmol) and PrI (14 μL, 0.14 mmol) were reacted under the same conditions and purified just as 14b to obtain 15d (21.7 mg, 43% yield) as an off-white solid. ¹H NMR (400 MHz, cdcl3) δ 0.99 (3H, t, J=7.4 Hz), 1.47 (6H, s), 1.99 (2H, dq, J=7.4, 14.7 Hz), 4.37 (2H, t, J=7.1 Hz), 5.15 (2H, s), 7.48 (2H, d, J=8.1 Hz), 7.76 (1H, s), 7.79-7.90 (2H, m), 7.90-8.00 (2H, m) ppm. ¹³C NMR (101 MHz, cdcl3) δ 179.88, 175.33, 137.31, 135.89, 135.15, 133.67, 133.32, 132.17, 130.77, 128.29, 127.08, 126.14, 123.23, 120.50, 119.60, 114.81, 110.10, 65.37, 52.08, 47.32, 23.75, 23.65, 11.06.

### Example 14

This example demonstrates a synthesis of 4-(3-(4-(1-butyl-1H-1,2,3-triazol-4-yl)benzyl)-4,4-dimethyl-5-oxo-2-thioxoimidazolidin-1-yl)-2-(trifluoromethyl)benzonitrile (15e).

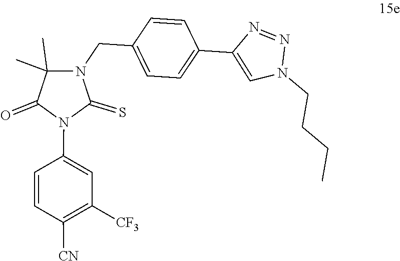 [SMILES: CCCCn1cc(-c2ccc(CN3C(=S)N(c4ccc(C#N)c(C(F)(F)F)c4)C(=O)C3(C)C)cc2)nn1]

Thiohydantoin benzyl alkyne 10 (40 mg, 0.094 mmol) and BuI (16 μL, 0.14 mmol) were reacted under the same conditions and purified just as 14b to obtain 15e (15.9 mg, 32.3% yield) as an off-white solid. ¹H NMR (400 MHz, cdcl3) δ 0.97 (3H, t, J=7.4 Hz), 1.40 (2H, sex, J=7.4 Hz), 1.47 (6H, s), 1.94 (2H, quin, J=7.4 Hz), 4.41 (2H, t, J=7.2 Hz), 5.16 (2H, s), 7.49 (2H, d, J=8.1 Hz), 7.75 (1H, s), 7.80-7.86 (3H, m), 7.95 (1H, d, J=2.0 Hz), 7.97 (1H, d, J=8.3 Hz) ppm. ¹³C NMR (101 MHz, cdcl3) δ 179.88, 175.33, 137.30, 135.87, 135.14, 133.69, 133.35, 132.15, 128.30, 127.08, 126.12, 123.23, 120.52, 114.80, 110.14, 65.35, 50.25, 47.33, 32.29, 23.66, 19.71, 13.45.

### Example 15

This example provides the NMR and mass spectrum data for various compounds of formula (I).

4-(3-(2-Fluoro-4-(1H-1,2,3-triazol-4-yl)benzyl)-4,4-dimethyl-2,5-dioxoimidazolidin-1-yl)-2-(trifluoromethyl)benzonitrile (19a). ¹H NMR (400 MHz, CDCl₃) (12.46-12.00 (bs, 1H), 8.15 (s, 1H), 8.01 (d, J=8.6 Hz, 1H), 7.98-7.88 (m, 2H), 7.57 (d, J=7.3 Hz, 3H), 4.71 (s, 2H), 1.46 (s, 6H). HRMS (ESI) m z Calcd. for C₂₂H₁₇N₆F₄O₂ [M+H⁺]: 473.1330, found 473.1344.

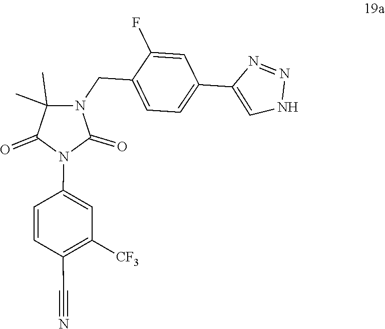 [SMILES: CC1(C)C(=O)N(c2ccc(C#N)c(C(F)(F)F)c2)C(=O)N1Cc1ccc(-c2c[nH]nn2)cc1F]

4-(3-(3-Fluoro-4-(1H-1,2,3-triazol-4-yl)benzyl)-4,4-dimethyl-2,5-dioxoimidazolidin-1-yl)-2-(trifluoromethyl)benzonitrile (19b). ¹H NMR (400 MHz, CDCl₃) (12.41 (bs, 1H), 8.11 (s, 1H), 8.07-7.92 (m, 3H), 7.87 (d, J=8.4 Hz, 1H), 7.16 (d, J=19.1 Hz, 2H), 4.58 (s, 2H), 1.40 (s, 6H). HRMS (ESI) m z Calcd. for C₂₂H₁₇N₆F₄O₂ [M+H⁺]: 473.1330, found 473.1344.

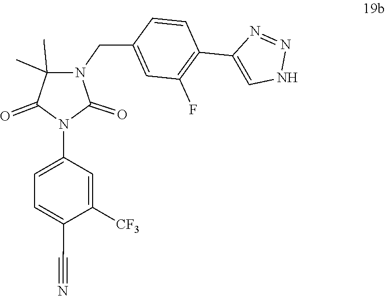 [SMILES: CC1(C)C(=O)N(c2ccc(C#N)c(C(F)(F)F)c2)C(=O)N1Cc1ccc(-c2c[nH]nn2)c(F)c1]

4-(3-(3-Fluoro-4-(1H-1,2,3-triazol-4-yl)benzyl)-4,4-dimethyl-5-oxo-2-thioxoimidazolidin-1-yl)-2-(trifluoromethyl)benzonitrile (19c). ¹H NMR (400 MHz, DMSO-d₆) δ 8.36 (dd, J=24.1, 10.1 Hz, 2H), 8.19 (s, 1H), 8.13 (d, J=7.8 Hz, 1H), 7.97 (d, J=11.0 Hz, 2H), 7.46 (dd, J=22.7, 9.7 Hz, 2H), 5.15 (s, 2H), 1.50 (s, 7H). HRMS (ESI) m z Calcd. for C₂₂H₁₇ON₆F₄S [M+H⁺]: 489.1104, found 489.1115.

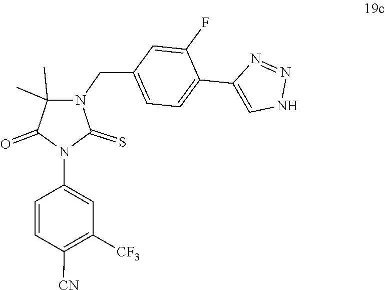 [SMILES: CC1(C)C(=O)N(c2ccc(C#N)c(C(F)(F)F)c2)C(=S)N1Cc1ccc(-c2c[nH]nn2)c(F)c1]

4-(3-(2-Fluoro-4-(1H-1,2,3-triazol-4-yl)benzyl)-4,4-dimethyl-5-oxo-2-thioxoimidazolidin-1-yl)-2-(trifluoromethyl)benzonitrile (19d). ¹H NMR (400 MHz, DMSO-d₆) δ 8.40 (d, J=8.1 Hz, 2H), 8.33 (d, J=7.6 Hz, 1H), 8.11 (d, J=8.0 Hz, 1H), 7.95 (s, 1H), 7.78-7.56 (m, 3H), 5.12 (s, 2H), 1.53 (s, 6H). HRMS (ESI) m z Calcd. for C₂₂H₁₇ON₆F₄S [M+H⁺]: 489.1102, found 489.1115.

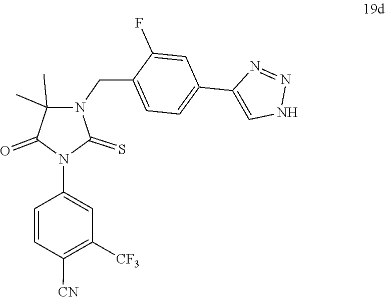 [SMILES: CC1(C)C(=O)N(c2ccc(C#N)c(C(F)(F)F)c2)C(=S)N1Cc1ccc(-c2c[nH]nn2)cc1F]

### Example 16

This example demonstrates a 4-(3-(4-azidobenzyl)-4,4-dimethyl-2,5-dioxoimidazolidin-1-yl)-2-(trifluoromethyl)benzonitrile (20).

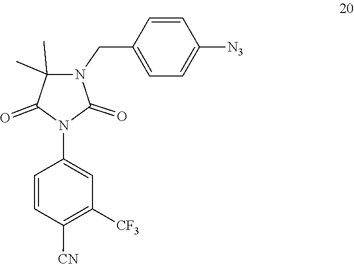 [SMILES: CC1(C)C(=O)N(c2ccc(C#N)c(C(F)(F)F)c2)C(=O)N1Cc1ccc(N=[N+]=[N-])cc1]

Cyano-nilutamide (4) (500 mg, 1.682 mmol) was dissolved in 5 mL of DMF under argon, followed by the addition of NaH (60% in mineral oil, 114.3 mg, 2.859 mmol) and allowed to react at room temperature for 2 hours. Mesylated p-azidobenzyl alcohol (548 mg, 2.412 mmol) was added, and the temperature was raised to 53° C. and stirred for 2 hours. The reaction mixture was allowed to cool to room temperature diluted with EtOAc and washed alternately with brine and water (3× each, 100 mL). The organic layer was dried over Na₂SO₄, filtered, and concentrated. The resulting residue was purified via column chromatography (eluent 3:1:0.1 Hex:EtOAc:Acetone→1:1 Hex:EtOAc) yielding 20 as an off-white solid (702 mg, 97% yield). ¹H NMR (400 MHz, cdcl3) δ 8.16 (d, J=2.1 Hz, 1H), 8.02 (dd, J=8.4, 2.1 Hz, 1H), 7.88 (d, J=8.5 Hz, 1H), 7.35 (d, J=8.7 Hz, 2H), 6.97 (d, J=8.6 Hz, 2H), 4.58 (s, 2H), 1.40 (s, 6H).

### Example 17

This example demonstrates a synthesis of 4-(3-(4-(1H-1,2,3-triazol-1-yl)benzyl)-4,4-dimethyl-2,5-dioxoimidazolidin-1-yl)-2-(trifluoromethyl)benzonitrile/4-(4,4-dimethyl-2,5-dioxo-3-(4-(4-(trimethylsilyl)-1H-1,2,3-triazol-1-yl)benzyl)imidazolidin-1-yl)-2-(trifluoromethyl)benzonitrile (20a), which is a representative procedure for synthesis of reverse triazole alcohols (20a-e).

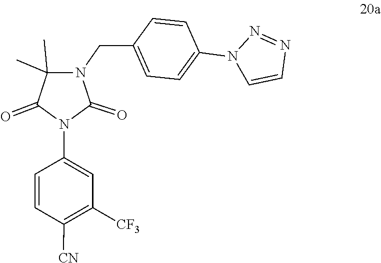 [SMILES: CC1(C)C(=O)N(c2ccc(C#N)c(C(F)(F)F)c2)C(=O)N1Cc1ccc(-n2ccnn2)cc1]

Hydantoin benzyl azide 20 (100 mg, 0.2334 mmol), TMS acetylene (25.22 mg, 0.2568 mmol), and DIPEA (60.33 mg, 0.4668 mmol) were dissolved in 2 mL of DMSO. Argon was bubbled through the solution for 20 minutes after which CuI was added (22.23 mg, 0.1167 mmol). Argon was bubbled through the solution for an additional 10 minutes after which it was lifted above the solution, and the solution was left to stir overnight. The reaction mixture was diluted with EtOAc and washed with 4:1 saturated NH₄Cl:NH₄OH (3×100 mL). The organic layer was dried over Na₂SO₄, filtered, and concentrated in vacuo. The residue was purified via preparative TLC (1:1 Hex:EtOAc). HRMS Theo: 455.1438 found 455.1436.

### Example 18

This example demonstrates a synthesis of 4-(3-(4-(4-(hydroxymethyl)-1H-1,2,3-triazol-1-yl)benzyl)-4,4-dimethyl-2,5-dioxoimidazolidin-1-yl) 2-(trifluoromethyl)benzonitrile (20b).

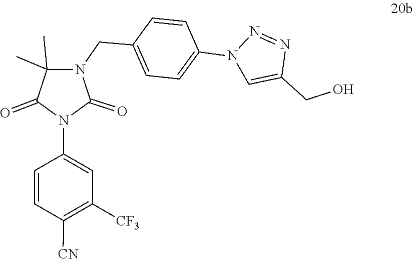 [SMILES: CC1(C)C(=O)N(c2ccc(C#N)c(C(F)(F)F)c2)C(=O)N1Cc1ccc(-n2cc(CO)nn2)cc1]

Hydantoin benzyl azide 20 (100 mg, 0.2334 mmol) and propargyl alcohol (23.55 mg, 0.4201 mmol) were reacted under the same conditions as 20a. The resulting crude material was purified via preparative TLC (6:1 EtOAc:Hex) to yield 20b as an off-white solid (94.5 mg, 84% yield). ¹H NMR (400 MHz, cdcl3) δ 8.18 (s, 1H), 8.04 (dd, J=8.4, 2.1 Hz, 1H), 7.98 (s, 1H), 7.94 (d, J=8.3 Hz, 1H), 7.74 (d, J=8.4 Hz, 2H), 7.55 (d, J=8.4 Hz, 2H), 4.90 (s, 2H), 4.69 (s, 2H), 1.59 (s, 1H), 1.47 (s, 6H). ¹³C NMR (101 MHz, cdcl3) δ 174.38, 153.40, 148.79, 137.81, 136.56, 136.36, 135.33, 133.93, 133.60, 133.27, 132.94, 129.35, 128.07, 126.02, 123.01, 120.80, 120.13, 115.00, 108.23, 64.37, 62.28, 56.14, 43.05, 23.63. HRMS Theo: 485.1543 found 485.1539.

### Example 19

This example demonstrates a synthesis of 4-(3-(4-(4-(2-hydroxyethyl)-1H-1,2,3-triazol-1-yl)benzyl)-4,4-dimethyl-2,5-dioxoimidazolidin-1-yl)-2-(trifluoromethyl)benzonitrile (20c).

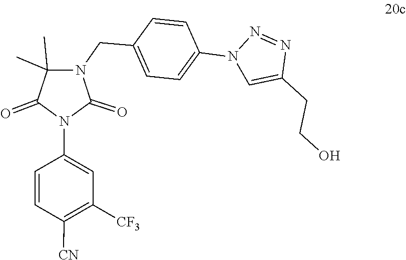 [SMILES: CC1(C)C(=O)N(c2ccc(C#N)c(C(F)(F)F)c2)C(=O)N1Cc1ccc(-n2cc(CCO)nn2)cc1]

Hydantoin benzyl azide 20 (60 mg, 0.1400 mmol) and 3-butynol (17.66 mg, 0.2521 mmol) were reacted under the same conditions as 20a. The resulting crude product was purified via preparative TLC (6:1 EtOAc:Hex) to yield 20c as an off-white solid (67 mg, 96% yield). ¹H NMR (400 MHz, cdcl3) δ 8.19 (d, J=2.1 Hz, 1H), 8.04 (dd, J=8.4, 2.1 Hz, 1H), 7.94 (d, J=8.4 Hz, 1H), 7.85 (s, 1H), 7.74 (d, J=8.5 Hz, 2H), 7.54 (d, J=8.4 Hz, 2H), 4.68 (s, 2H), 4.02 (t, J=5.6 Hz, 2H), 3.04 (t, J=5.8 Hz, 2H), 1.46 (s, 6H). HRMS Theo: 499.1700 found 499.1696

### Example 20

This example demonstrates a synthesis of 4-(3-(4-(4-(3-hydroxypropyl)-1H-1,2,3-triazol-1-yl)benzyl)-4,4-dimethyl-2,5-dioxoimidazolidin-1-yl)-2-(trifluoromethyl)benzonitrile (20d).

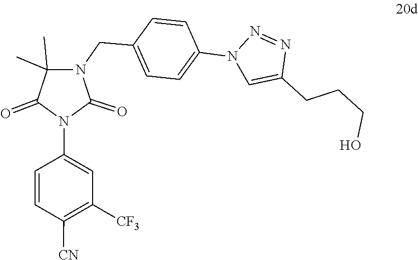 [SMILES: CC1(C)C(=O)N(c2ccc(C#N)c(C(F)(F)F)c2)C(=O)N1Cc1ccc(-n2cc(CCCO)nn2)cc1]

Hydantoin benzyl azide 20 (60 mg, 0.1400 mmol) and 4-pentynol (21.20 mg, 0.2521 mmol) were reacted under the same conditions as 20a. The resulting crude material was purified via preparative TLC (EtOAc) to yield 20d as an off-white solid (71.3 mg, 94.6% yield). ¹H NMR (400 MHz, cdcl3) δ 8.19 (d, J=1.9 Hz, 1H), 8.04 (dd, J=8.4, 2.1 Hz, 1H), 7.94 (d, J=8.4 Hz, 1H), 7.76 (s, 1H), 7.73 (d, J=8.5 Hz, 2H), 7.54 (d, J=8.5 Hz, 2H), 4.68 (s, 2H), 3.76 (t, J=5.1 Hz, 2H), 2.93 (t, J=7.2 Hz, 2H), 2.01 (s, 2H), 1.46 (s, 6H). HRMS Theo: 513.1856 found 513.1853.

### Example 21

This example demonstrates a synthesis of 4-(3-(4-(4-(4-hydroxybutyl)-1H-1,2,3-triazol-1-yl)benzyl)-4,4-dimethyl-2,5-dioxoimidazolidin-1-yl)-2-(trifluoromethyl)benzonitrile (20e).

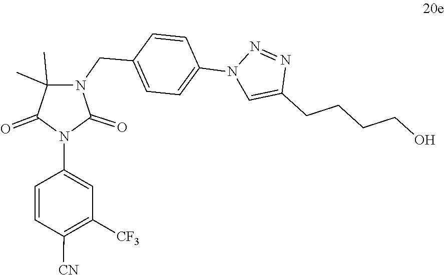 [SMILES: CC1(C)C(=O)N(c2ccc(C#N)c(C(F)(F)F)c2)C(=O)N1Cc1ccc(-n2cc(CCCCO)nn2)cc1]

Hydantoin benzyl azide 20 (60 mg, 0.1400 mmol) and 5-hexynol (24.74 mg, 0.2521 mmol) were reacted under the same conditions as 20a. The resulting crude material was purified via preparative TLC (EtOAc) to yield 20e as an off-white solid (57.3 mg, 74% yield). ¹H NMR (400 MHz, cdcl3) δ 8.19 (d, J=2.0 Hz, 1H), 8.04 (dd, J=8.5, 1.8 Hz, 1H), 7.94 (d, J=8.4 Hz, 1H), 7.75 (t, J=12.3 Hz, 3H), 7.53 (d, J=8.4 Hz, 2H), 4.68 (s, 2H), 3.71 (t, J=6.1 Hz, 2H), 2.85 (t, J=6.9 Hz, 2H), 1.90-1.79 (m, 2H), 1.73-1.64 (m, 2H), 1.59 (s, 1H), 1.46 (s, 6H). HRMS Theo: 527.2013 found 527.2011.

### Example 22

This example demonstrates the biological activity of exemplary compounds of formula (I).

Example compound structure activity relationship in an assay of AR transcriptional output, performed in HEK 293T cells co-transfected with a firefly luciferase under the control of an AR response element, and also a pCMXβGal to express β-galactosidase as an internal control. Cells were maintained in steroid free media, and stimulated with 200 μM testosterone, followed immediately by administration of exemplary compounds (Table 1, below). RLU is relative luciferase units, relative to DMSO control with 200 μM testosterone alone. Data represent an average of 3 independent experiments performed in triplicate wells per compounds at 10 μM.

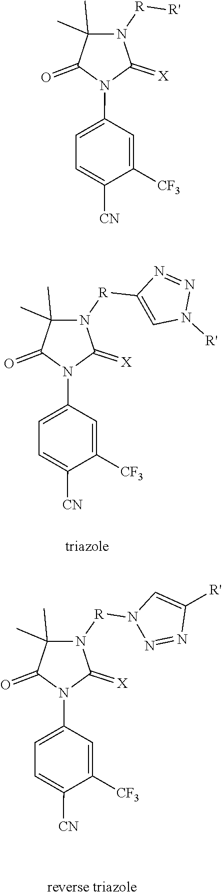

TABLE 1

| | | | | | AR antagonist |
|---|---|---|---|---|---|
| analogue | class | R | R′ | X | activity (% RLU) |
| 6 | alkynes | benzyl | alkyne | O | 138% |
| 10 | | benzyl | alkyne | S | 95% |
| 12 | | methyl | alkyne | S | 546% |
| 13 | | Ph | alkyne | S | 59% |
| 14a | triazoles | benzyl | H | O | −64% |
| 14b | | benzyl | Me | O | −3% |
| 15a | | benzyl | H | S | −109% |
| 15b | | benzyl | Me | S | −41% |
| 15c | | benzyl | Et | S | 3% |
| 15d | | benzyl | Pr | S | 4% |
| 15e | | benzyl | Bu | S | −3% |
| 16a | | Ph | H | S | −22% |
| 18a | | methyl | H | S | 412% |
| 20a | | benzyl | H | O | 57% |
| 20b | reverse | benzyl | MeOH | O | 73% |
| 20c | triazoles | benzyl | EtOH | O | −1% |
| bicalutamide | | | | | 9% |

### Example 23

This example demonstrates the AR binding affinity of exemplary compounds of formula (I).

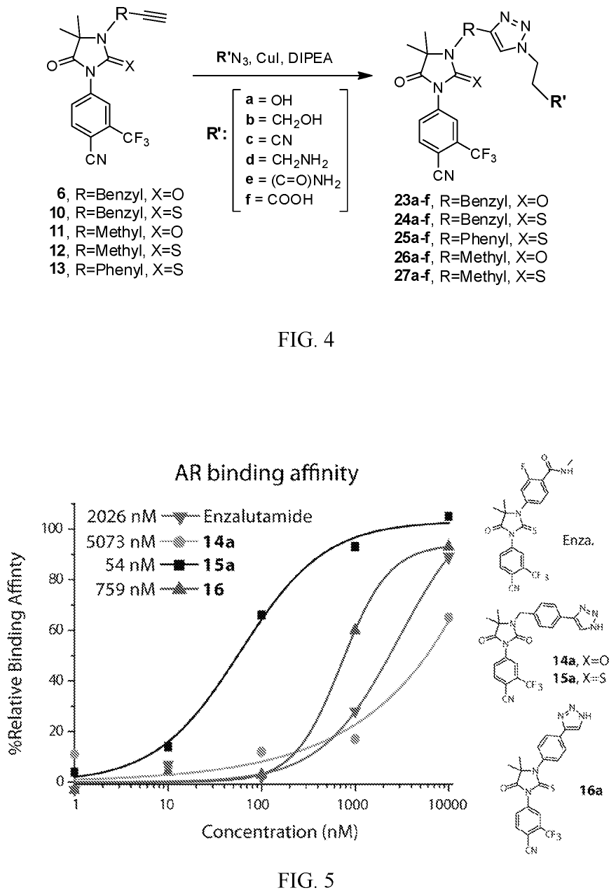

Radioligand binding (Eurofins Panlabs, St. Charles, Mo.) was performed using rat androgen receptor (AR) and [³H] mibolerone (PanVera, Madison, Wis.) in triphosphate buffer (pH 7.4). 78 ng of AR was incubated with 1.5 nM [³H] mibolerone for 4 h at 4° C., then incubated with a hydroxyapatite slurry over 15 minutes and filtered. The filters were washed 3 times and counted to determine [³H] mibolerone specifically bound. Compounds were tested in a logarithmic concentration range from 10 nM to 100 μM to determine Kᵢ and IC₅₀ values. The results are shown in FIG. 5.

### Example 24

This example demonstrates the inhibition of AR transcriptional activity by exemplary compounds of formula (I).

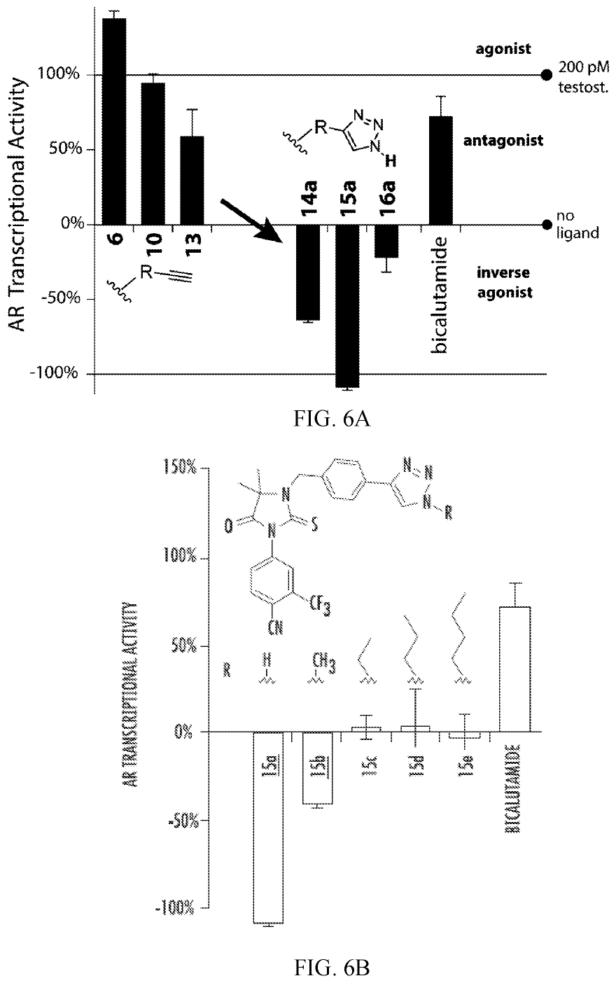

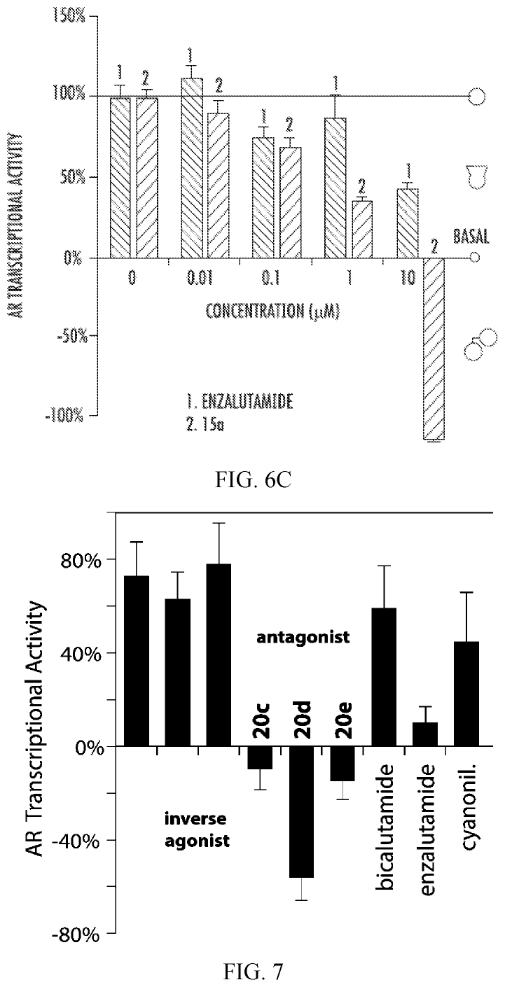

AR transcriptional output was measured in HEK 293T cells co-transfected with a firefly luciferase under the control of an AR response element, and also a pCMXβGal to express β-galactosidase as an internal control. Cells were maintained in steroid-free media, and stimulated with 200 μM testosterone, followed immediately by administration of a test compound. FIG. 6A shows the antagonist or inverse agonist activity of 6, 10, 13, 14a, 15a, 16a, and bicalutamide. FIG. 6B shows the antagonist or inverse agonist activity of 15a, 15b, 15c, 15d, 15e, and bicalutamide. Compounds were dosed at 10 μM in FIGS. 6A and 6B. FIG. 6C shows the antagonist or inverse agonist activity of 15a and enzalutamide at varying concentrations (M).

The first set of 1H-triazole analogues showed a substantial switch from their alkyne precursors 6, 10, and 13, which were relatively weak antagonists (FIG. 6A). The triazole compounds 14a, 15a, and 16 showed a surprising feature in that at 10 μM they reduced the transcriptional activity of the AR well below the expression in the absence of any steroids or ligands (cells were cultured in charcoal stripped serum, which have levels of testosterone less than 0.02 ng/mL, below standard detection limits) (Cao et al., Endocrine Research, 2009, 34(4), 101-108). Since molecular docking to the AR left the 1H-triazole as the outward facing, solvent exposed portion of the ligand, it was suspected the lead (15a) could be destabilized by adding a series of alkyl linkers (FIG. 6B).

### Example 25

This example demonstrates the inhibition of AR transcriptional activity by exemplary compounds of formula (I).

AR transcriptional output was measured in HEK 293T cells co-transfected with a firefly luciferase under the control of an AR response element, and also a pCMXβGal to express β-galactosidase as an internal control. Cells were maintained in steroid-free media, and stimulated with 200 μM testosterone, followed immediately by administration of a test compound. FIG. 7 shows the antagonist or inverse agonist activity of 20, 20a-e, bicalutamide, enzalutamide, and cyanonilutamide.

### Example 26

This example demonstrates the cytotoxicity of exemplary compounds of formula (I).

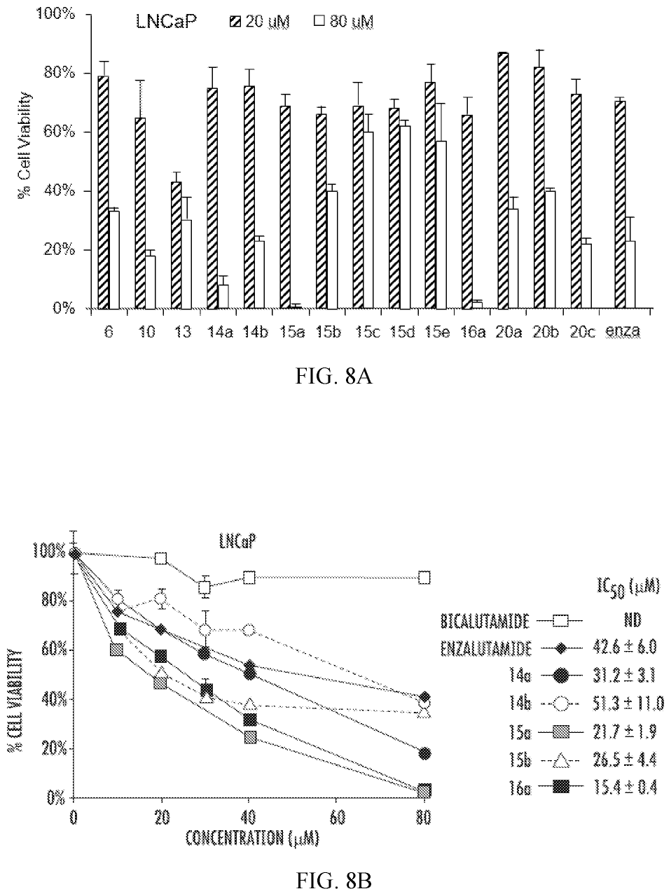

Cells were grown at 37° C. under a 5% CO₂ atmosphere in 10% FBS and RPMI culture media in 96 well plates. Cells were then treated with indicated concentrations of test compound, followed by colorimetric MTT (3-(4,5-dimethylthiazol-2-yl)-2,5-diphenyltetrazolium bromide) assay to quantify impact on cell viability relative to control wells. The ability for compounds to kill AR-positive prostate cancer cells, LNCaP, are shown in FIG. 8A and FIG. 8B at varying doses.

### Example 27

This example demonstrates the cytotoxicity of exemplary compounds of formula (I).

Cells were grown in DMEM (Dulbecco's Modified Eagle Medium) with 10% FBS (fetal bovine serum) in 384 well plates for 1 day, then dosed with indicated concentrations of exemplary compounds, followed by imaging of all wells every 4 hours by INCUCYTE™ ZOOM (Sartorius, Goettingen, Germany). Images were then analyzed to discern the % confluence of cells in each well. IC₅₀ values were calculated from dose response curves of the % cell growth inhibition relative to DMSO control at the same time point per each cell line. The IC₅₀ values (μM) for various AR-positive (LNCaP, LNCAPAR, LAPC4, 22Rv1, LNCaP AR_{in vivo}, and LNCaP ARF876L) and AR-negative (PC3 and DU145) prostate cancer cell lines are shown in Table 2.

TABLE 2
IC₅₀ values (μM)

| | | AR-Positive | | | | | | | |
|---|---|---|---|---|---|---|---|---|---|
| | | AR Mutation | | | | | | | |
| | | T877A | w.t. | w.t. | H874Y | w.t. | F876L | AR-Negative | |
| | | Cell Line | | | | | | | |
| | | | LNCAP | | | LNCaP | LNCaP | | |
| | | LNCaP | AR | LAPC4 | 22Rv1 | AR_{in vivo} | AR_{F876L} | PC3 | DU145 |
| Compound | 14a | 44.8 | 32.5 | 70.0 | 46.5 | 65.4 | 35.9 | 68.5 | 84.1 |
| | 19a | 39.0 | 29.4 | 30.7 | 36.7 | 35.8 | 41.4 | 63.4 | 46.1 |
| | 19b | 43.1 | 35.1 | 33.1 | 40.6 | 60.1 | 49.4 | 60.9 | 65.0 |
| | 15a | 30.5 | 17.0 | 13.3 | 18.3 | 18.5 | 21.0 | 59.8 | 37.3 |
| | 16a | 17.1 | 21.2 | 17.3 | 20.7 | 33.6 | 30.2 | 49.2 | 39.9 |
| | 19c | 36.2 | 28.0 | 17.9 | 25.3 | 34.0 | 33.9 | 59.9 | 114.2 |
| | 19d | 35.7 | 28.2 | 16.3 | 26.9 | 28.2 | 22.3 | 44.8 | 53.6 |
| | Enzalutamide | 59.0 | 53.5 | 67.9 | 70.8 | 92.7 | 85.9 | 199.9 | 86.3 |
| | ARN509 | 34.5 | 98.9 | 86.0 | 32.5 | 270.2 | 98.7 | 300.0 | 61.8 |
| | AZD3514 | 76.9 | 25.8 | 30.5 | 26.8 | | | 121.6 | 61.2 |
| | Bicalutamide | | 80.5 | 67.0 | 53.4 | | | 239.4 | |

As seen in Table 2, compounds of formula (I) are inverse agonists more potent than enzalutamide. Importantly, the triazole series was not hindered by the AR mutations that limit enzalutamide and ARN-509 (Korpal et al., Cancer Discovery, 2013, 3(9), 1030-1043).

### Example 28

This example demonstrates the ability of an exemplary compound of formula (I) to inhibit growth of prostate cancer cells.

Percent confluence was measured via phase-contrast images of cell in culture using the INCUCYTE™ ZOOM live cell imaging system (Sartorius, Goettingen, Germany). Data for each time-point shown is an average of 4 replicates.

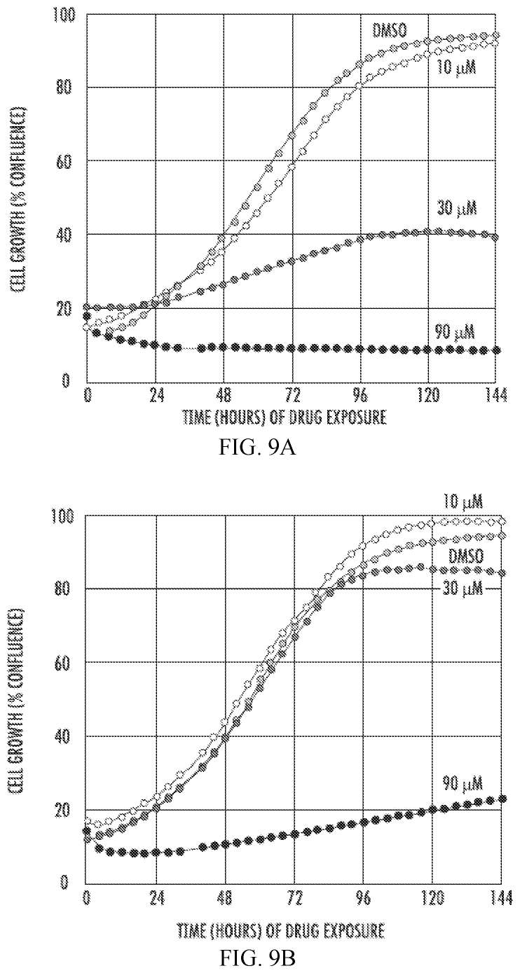

FIGS. 9A and 9B show the impact of 19c on the growth of AR-positive prostate cancer cell line LNCaP-AR (FIG. 9A), compared to enzalutamide (FIG. 9B).

### Example 29

This example demonstrates the dose response of 16a and 19c, exemplary compounds of formula (I).

Percent confluence was measured via phase-contrast images of cell in culture using the INCUCYTE™ ZOOM live cell imaging system (Sartorius, Goettingen, Germany). Data for each time-point shown is an average of 4 replicates.

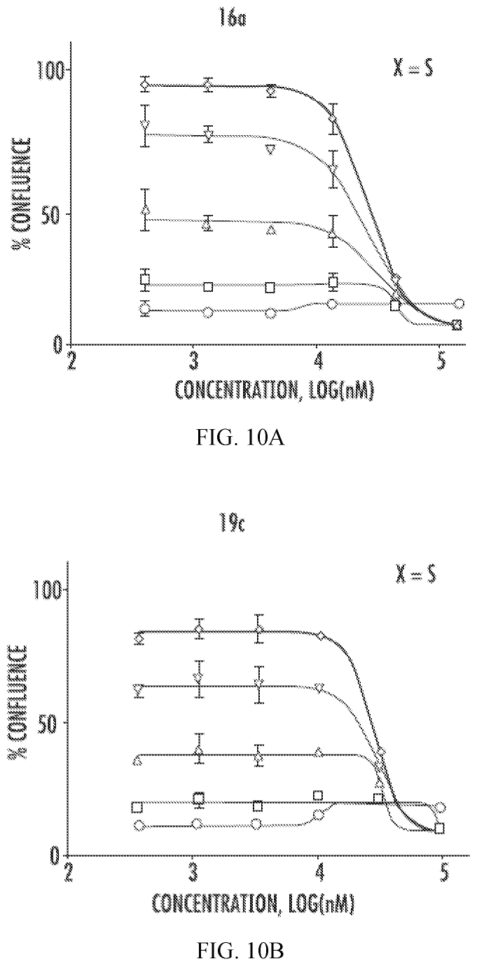

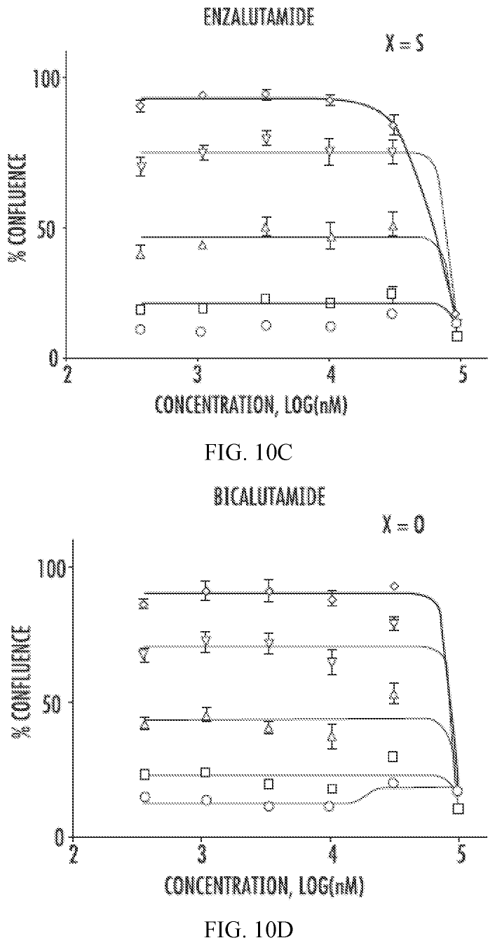

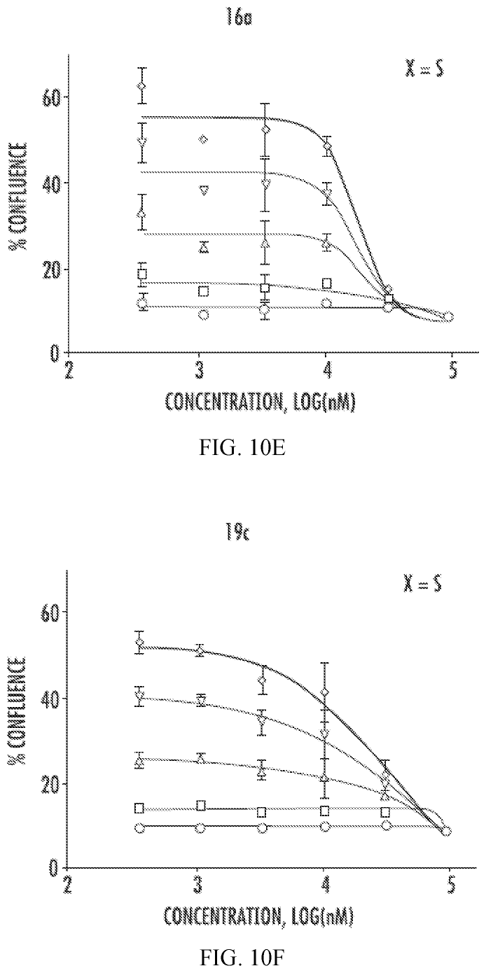

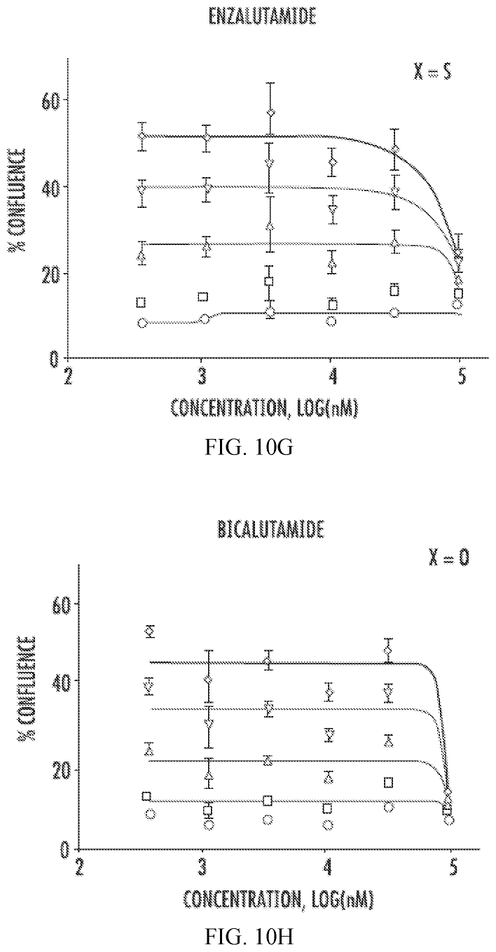

The results for 16a, 19c, enzalutamide, and bicalutamide are shown for AR+ prostate cancer cell lines in FIGS. 10A-10D (LNCaP-AR) and FIGS. 10E-10H (LAPC4).

### Example 30

This example demonstrates the in vitro binding of exemplary compounds of formula (I) to various receptors.

Binding assays were performed via competition with radioactive ligands across a range of dose of compounds 15a and 19d. For aldosterone binding, the aldosterone receptor was incubated for 20 hours with 0.4 [³H] aldosterone. Similarly, the androgen receptor was co-incubated with 0.5 nM of [³H] methyltrienolone, the estrogen receptor alpha was co-incubated with 0.5 nM [³H] estradiol, the glucocorticoid receptor with 0.5 nM [³H] dexamethasone, and the progesterone receptor with 0.5 nM [³H] progesterone as parent ligands. The results are shown in Table 3.

TABLE 3

Binding IC₅₀ (nM)

| Receptor | 15a | 19d |
|---|---|---|
| Aldosterone Receptor | >10,000 | >10,000 |
| Androgen Receptor | 12.5 | 9.3 |
| Estrogen Receptor alpha | >10,000 | >10,000 |
| Glucocorticoid Receptor | >10,000 | >10,000 |
| Progesterone Receptor B | 102.7 | 76.3 |

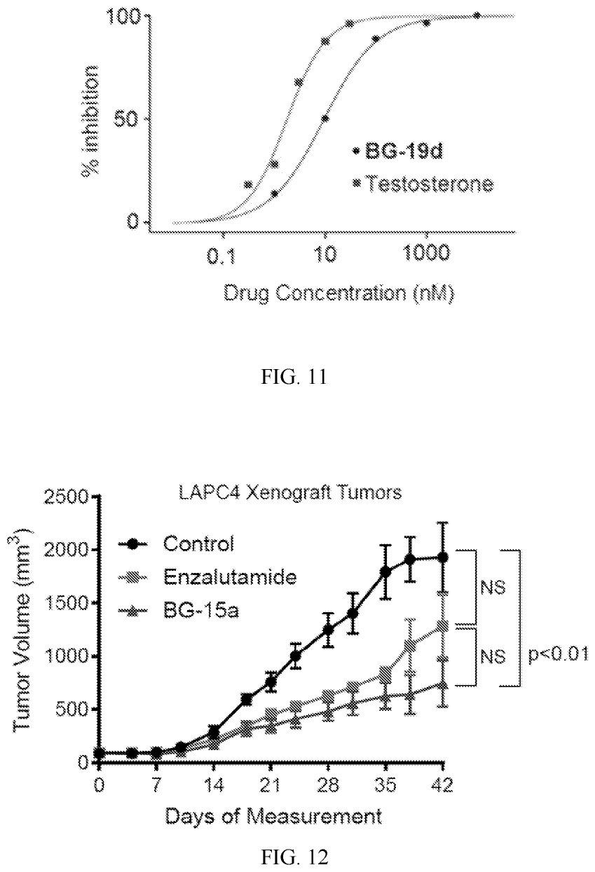

The AR binding of 19d compared to testosterone is plotted as drug concentration (nM) versus % inhibition in FIG. 11.

### Example 31

This example demonstrates the in vivo activity of 15a, an exemplary compound of formula (I).

Molecules were dissolved in Labrasol:saline (1:5 v/v) with 5% sucrose, given by oral gavage (100 μL). Mice were treated once daily for 5 days/week, resting for 2 days/week. Cohort size per treatment (n=8) is calculated by power analysis with a type I error of 5% and 80% power and effect size of 1.3. The in vivo activity is shown in FIG. 12, as measured by days of measurement versus tumor volume (mm³).

### Example 32

This example demonstrates the modification of 15a to provide a more water soluble derivative.

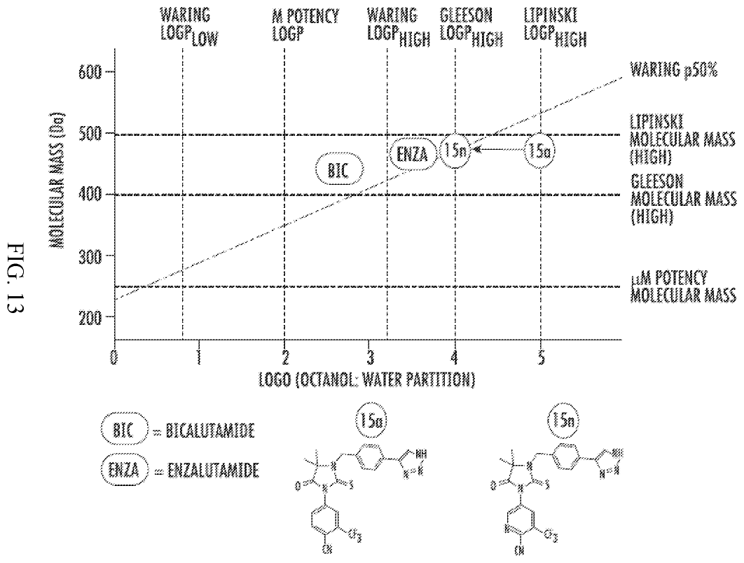

It was determined that compound 15a had an aromatic ring with the cyano group that sits in the base of the AR ligand binding domain, which could tolerate the addition of a nitrogen to the ring without impacting binding to the pocket, but which would add additional water solubility to the molecule by adding a hydrogen bond donor. The solubility improvement was predicted by log P (octanol to water partition coefficient). Drug characteristics (molecular mass and log P) of bicalutamide, enzalutamide, 15a, and 15n were calculated using ChemBioDraw (CambridgeSoft). The results in FIG. 13 show a 10-fold improvement of the octanol to water partition coefficient.

### Example 33

This example demonstrates the pharmacokinetics of 15a compared to 15n, an exemplary compound of formula (I).

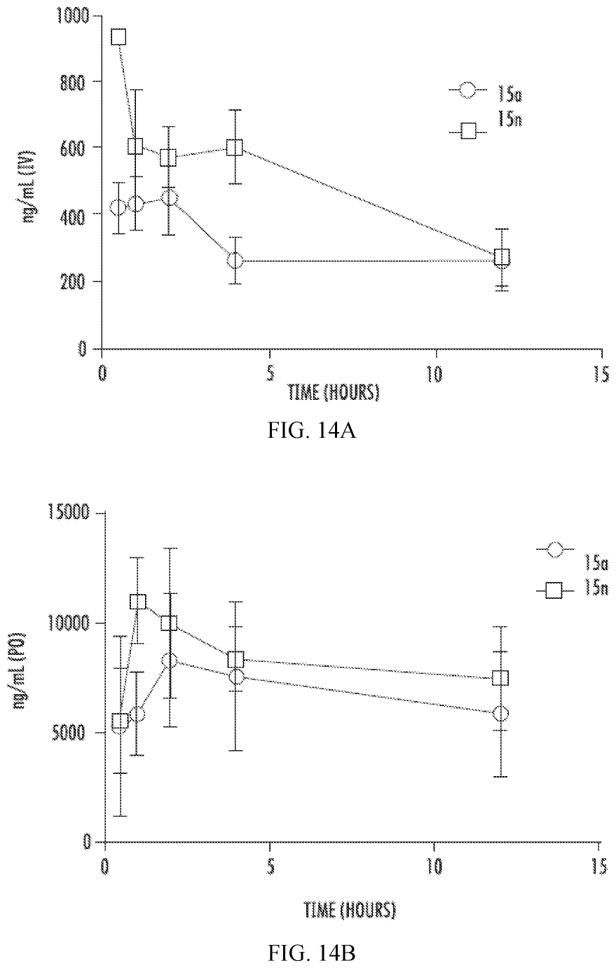

To rigorously evaluate the effect of the chemical modification from 15a to 15n, bioavailability was measured in non-tumor bearing BALB/C mice from blood drawn at 5 minutes, 30 minutes, 1 h, 2 h, 8 h, and 12 h post-injection of 15a or 15n by intravenous (IV) tail vein compared to oral (PO) gavage, by cardiac puncture, centrifuged and stored at −80° C. until analysis by mass spectrometry in triplicate. Clearance, Cₘₐₓ, half life, dose/AUC, and bioavailability were calculated by statistical analysis using PHOENIX™ WINNONLIN™ v7.0 (Certara, Princeton, N.J.). The results are shown in FIGS. 14A and 14B and Table 4.

TABLE 4

| | Compound | | | |
|---|---|---|---|---|
| | 15a | | 15n | |
| Route | IV | PO | IV | PO |
| # Samples | 5 | 5 | 5 | 5 |
| Dose (mg/kg) | 1 | 20 | 1 | 20 |
| T_{1/2} (hr) | 18.0 | 20.6 | 9.13 | 22.32 |
| Tₘₐₓ (hr) | 2.0 | 2.0 | 0.5 | 1.0 |
| Cₘₐₓ (ng/mL) | | | 936.94 | 10,981.75 |
| SE_Cₘₐₓ (ng/mL) | | | 7.75 | 1124.90 |
| Cₘₐₓ_{_{—}}D (kg*ng/mL/mg) | 453 | 412 | 936.94 | 549.09 |
| AUCₗₐₛₜ (hr*ng/mL) | | | 6255.85 | 97,329.66 |
| SE_AUCₗₐₛₜ (hr*ng/mL) | | | 397.02 | 7562.49 |
| AUCₗₐₛₜ_{_{—}}D (hr*kg* | | | 6255.85 | 4866.48 |
| ng/mL/mg) | | | | |

### Example 34

This example demonstrates a radioligand binding study of 15n, an exemplary compound of formula (I).

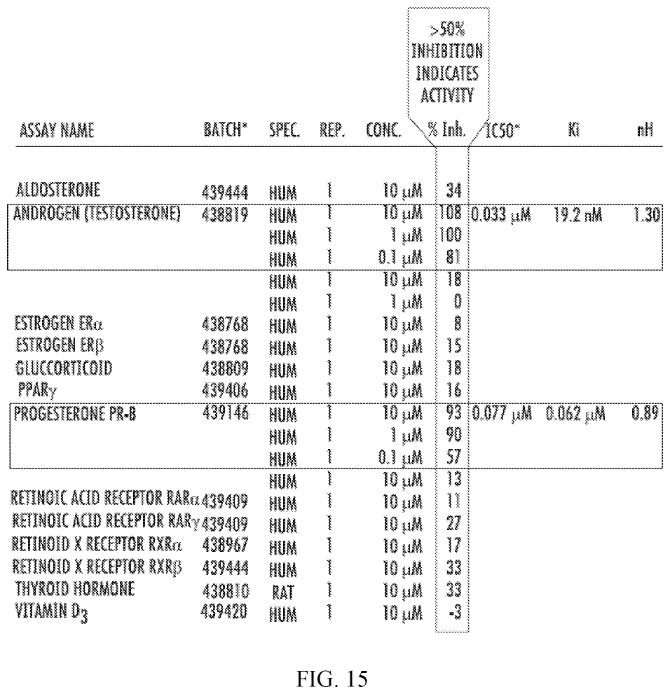

Radioligand binding of 15n in triphosphate buffer (pH 7.4) with 78 ng of AR and 1.5 nM [³H] mibolerone for 4 h at 4° C., then incubated with a hydroxyapatite slurry over 15 minutes and filtered, washed 3 times, and counted to determine [3H] mibolerone specifically bound after competition with indicated concentrations of 15n. The results are shown in FIG. 15.

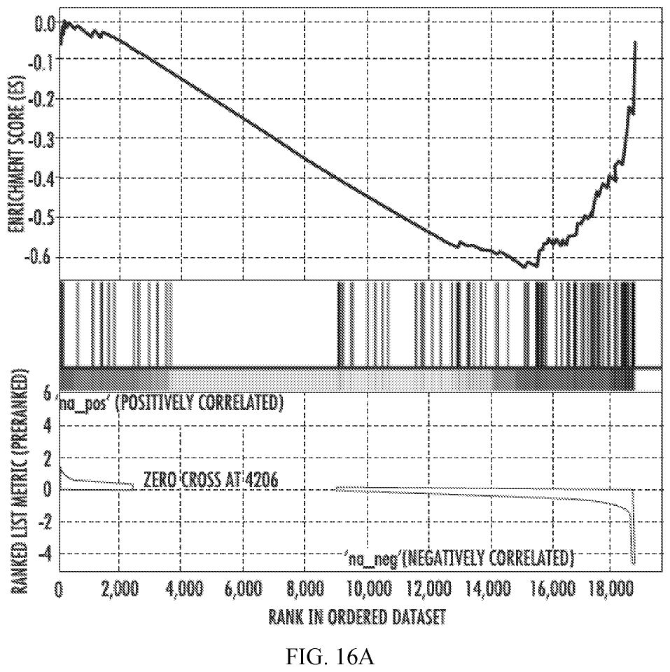

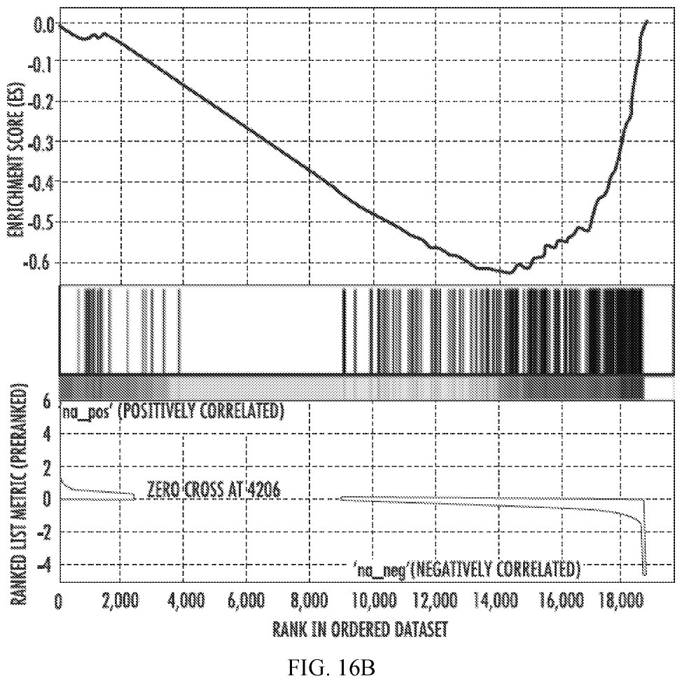

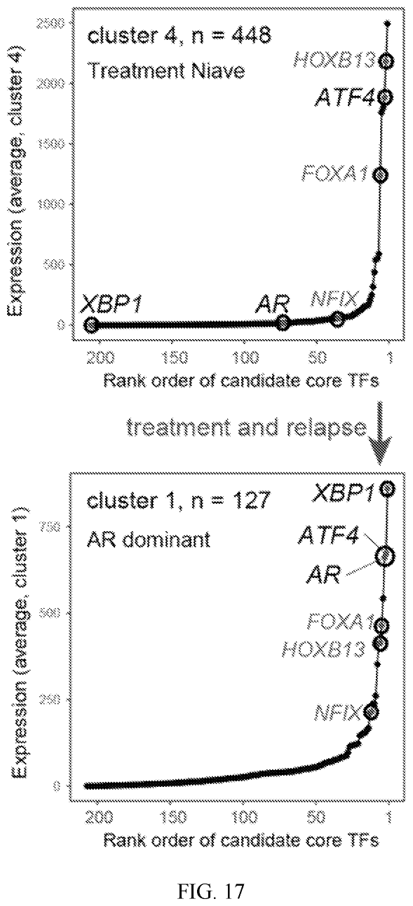

FIG. 16A-B are Gene Set Enrichment Analyses for AR gene pathways (FIG. 16A) and MYC gene pathways (FIG. 17B) in LAMP cells (LNCaP cells engineered with CRISPR (clustered regularly interspaced short palindromic repeats) to have amplification of the AR enhancer) treated with either 15n or DMSO for 48 hours, followed by RNA-seq. With a log 2-fold change, both the AR target gene set (FIG. 16A) and MYC target gene set (FIG. 16B) are shown to be down regulated with 15n.

### Example 35

This example demonstrates the impact of 15n, an exemplary compound of formula (I), on mCRPC models.

To evaluate the impact of 15n on mCRPC models, this group of diseases was defined in terms of transcriptional networks. There is strong transcriptional commitment to a small set of transcription factors (TFs), expressed at hundreds of times higher than the average TF, in each cancer subtype that define its identity. These “master TFs” or core regulatory TFs are drivers and inform gene networks essential to the disease state. To identify these in prostate cancer, statistical analysis was performed on public RNA-seq data (combining treatment naïve TCGA PCa and metastatic/relapsed PCa SU2C-PCF samples (Narayanan et al., Cancer Res., 2010, 70(2), 842-851)) as well as unpublished RNA-seq of advanced PCa PDX models. TFs which (1) are transcribed at unusually high levels in any subset of PCa samples and (2) are unique to PCa as compared to normal tissues and all other cancer types in TCGA were then (3) grouped by hierarchical clustering. TFs expression thus defined 4 subgroups of PCa. A key feature of relapse was evidenced in the transition from cluster 4 to cluster 1 (FIG. 17), where relapsed mCRPCs had overly high expression of three key TFs: AR, ATF4 and XBP1.

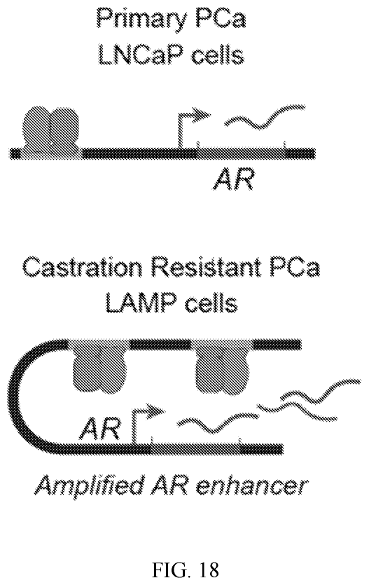

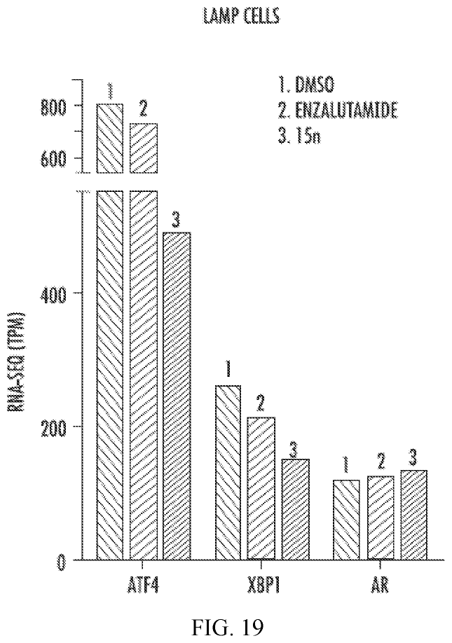

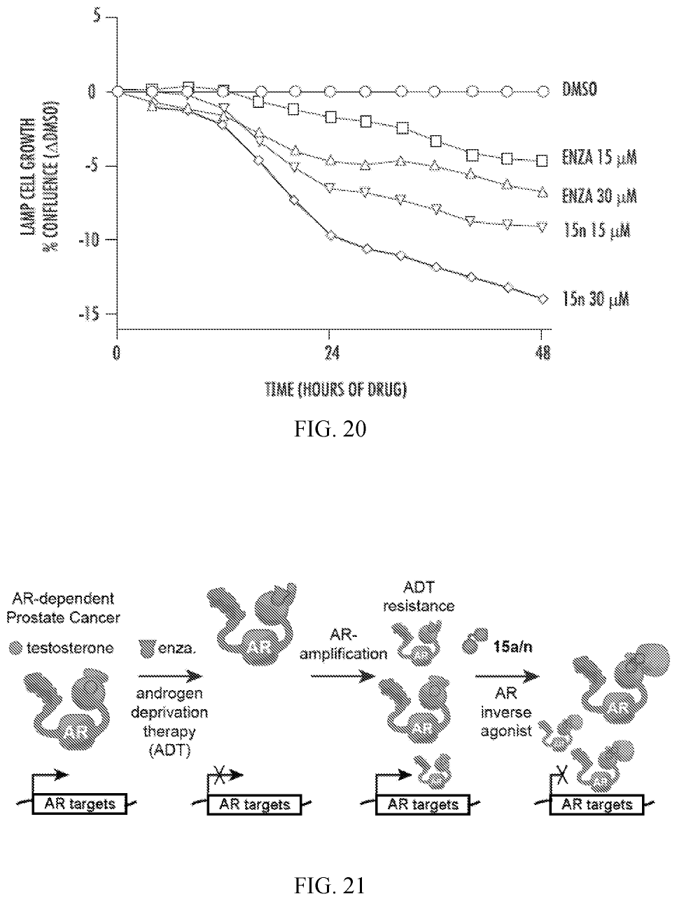

This AR dominated cluster 1 is often driven by amplification of the distal AR enhancer (Takeda et al., Cell, 2018, 174(2), 422-432). Thus, to model the distinct genome-wide differences between 15n and enzalutamide, LAMP cells (LNCaP given resistance to enzalutamide by CRISPR-knock in of AR enhancer amplification) (FIG. 18) were used. While both antagonist and inverse agonist equally downregulated AR target genes, only inverse agonist 15n was able to also downregulate the ATF4 driven amino-acid tRNA synthesis pathway. ATF4, the most highly expressed master TF in LAMP cells, was itself greatly reduced by 15n (FIG. 19). This may in part explain how 15n provides superior cell killing at lower doses than enzalutamide (FIG. 20) as ATF4 enables high levels of protein synthesis in support of MYC driven oncogenesis (Tameire et al., Nature Cell Biology, 2019, 21(7), 889-899). Thus, while AR overexpression can circumvent the traditional antagonist enzalutamide, 15n can work in this context to shutdown multiple TFs essential for the transcriptional output of mCRPC (FIG. 21).

### Example 36

This example demonstrates the in vivo activity of 15n, an exemplary compound of formula (I).

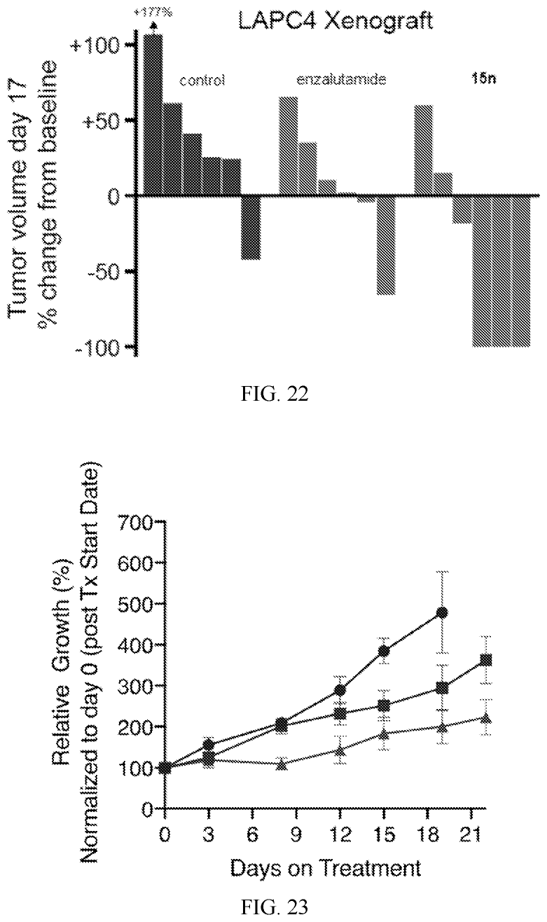

Mice were injected with LAPC4 prostate cancer cells and monitored until tumors were evident, then animals were treated with either vehicle, enzalutamide, or 15n via oral gavage at the indicated concentrations. Treatment was given once for 5 days followed by 2 days of rest. Tumor size was monitored by caliper twice weekly. Change from baseline tumor volume was calculated and reported as a waterfall plot. The in vivo activity is shown in FIG. 14, as measured by the percent change from the baseline. As seen in FIG. 22, 15n retained its ability to reduce tumor burden in LAPC4 mouse xenografts.

### Example 37

This example demonstrates the in vivo activity of 15n, an exemplary compound of formula (I).

LuCaP 167 is a well-characterized androgen sensitive prostate cancer (PCa) PDX model established at the University of Washington and has been validated using short tandem repeat (STR) analysis. All preclinical in vivo experiments were performed in accordance with NCI Animal Care and Use Committee approved protocol. Six-week-old NSGmale mice were housed in a sterile, pathogen-free facility and maintained in a temperature-controlled room under a 12-hour light/dark schedule with water and food ad libitum. All mice were operated under sedation with oxygen and isoflurane. Ibuprofen and/or buprenorphine was administered post-surgery. LuCaP 167 was implanted subcutaneously under the left flank of intact mice. When tumors reached an average volume of ˜500 mm³, mice were randomized and placed in either control group or treatment groups (n=10/group). Mice received enzalutamide treatment (10 mg/kg 5 days on 2 days off; oral gavage), 15n treatment (10 mg/kg 5 days on 2 days off, oral gavage) or vehicle (control). Tumor burden was assessed twice per week by caliper measurement of two diameters of the tumor (L×W=mm²) and reported as tumor volume ((L×W²)/2=mm³). Body weights were assessed using a weighing scale and recorded in grams. At the end-of-treatment (EOT) timepoint, tumor tissues were excised, weighed, and snap frozen for further analysis. FIG. 23 shows the average tumor growth rate per group±SEM, in which 15n had a superior anti-tumor effect over enzalutamide.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A compound of formula (I) 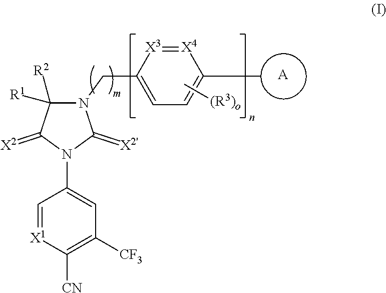
wherein
R¹ and R² are the same or different and each is alkyl,
X¹ is CH or N,
X² and X^{2′} are the same or different and each is O or S,
X³ and X⁴ are the same or different and each is CR³ or N,
R³ is hydrogen, halo, hydroxy, alkyl, or alkoxy,
ring A is selected from the group consisting of 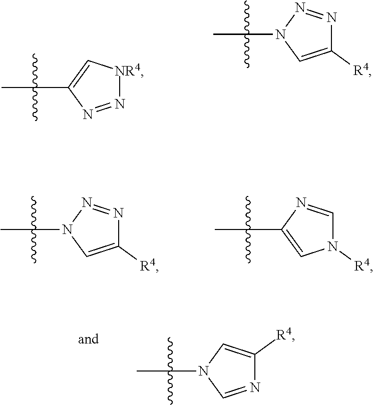
R⁴ is hydrogen, alkyl, cycloalkyl, heterocycloalkyl, aryl, or heteroaryl, wherein the alkyl, cycloalkyl, heterocycloalkyl, aryl, or heteroaryl is optionally substituted,
m is 0-8, n is 0 or 1, such that 1≤m+n≤9, and o is 0-2,
provided that when X¹ is CH, X³ and X⁴ are each CR³, ring A is 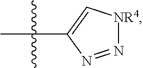
R⁴ is hydrogen or alkyl, n is 1, and o is 0-2, then at least one R³ is not hydrogen, or a pharmaceutically acceptable salt thereof.

2. The compound of claim 1, wherein R¹ and R² are each methyl, or a pharmaceutically acceptable salt thereof.

3. The compound of claim 1, wherein X¹ is N, or a pharmaceutically acceptable salt thereof.

4. The compound of claim 1, wherein X¹ is CH, or a pharmaceutically acceptable salt thereof.

5. The compound of claim 1, wherein X³ and X⁴ are each CR³, or a pharmaceutically acceptable salt thereof.

6. The compound of claim 1, wherein the compound of formula (I) is a compound of formula (Ia) 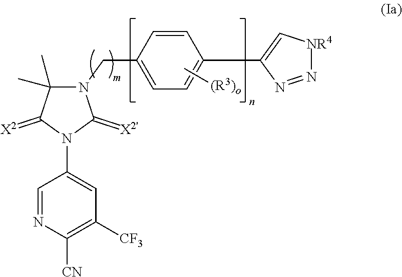
wherein
X² and X^{2′} are the same or different and each is O or S,
R³ is hydrogen, halo, hydroxy, alkyl, or alkoxy,
R⁴ is hydrogen, alkyl, cycloalkyl, heterocycloalkyl, aryl, or heteroaryl, wherein the alkyl, cycloalkyl, heterocycloalkyl, aryl, or heteroaryl is optionally substituted,
m is 0-8, n is 0 or 1, such that 1≤m+n≤9, and o is 0-2,
or a pharmaceutically acceptable salt thereof.

7. The compound of claim 1, wherein at least one R³ moiety is fluoro, or a pharmaceutically acceptable salt thereof.

8. The compound of claim 1, wherein X² is O and X^{2′} is S, or a pharmaceutically acceptable salt thereof.

9. The compound of claim 1, wherein X² and X^{2′} are O, or a pharmaceutically acceptable salt thereof.

10. The compound of claim 1, wherein m is 1, or a pharmaceutically acceptable salt thereof.

11. The compound of claim 1, wherein n is 1, or a pharmaceutically acceptable salt thereof.

12. The compound of claim 1, wherein R⁴ is hydrogen, or a pharmaceutically acceptable salt thereof.

13. The compound of claim 1, wherein R⁴ is alkyl that is optionally substituted, or a pharmaceutically acceptable salt thereof.

14. The compound of claim 13, wherein the alkyl is substituted with at least one substituent selected from hydroxyl, cyano, amino, alkylamino, dialkylamino, halo, nitro, carboxy, amido, cycloalkyl, heterocycloalkyl, aryl, and heteroaryl, or a pharmaceutically acceptable salt thereof.

15. The compound of claim 1, wherein R³ is not hydrogen, that is 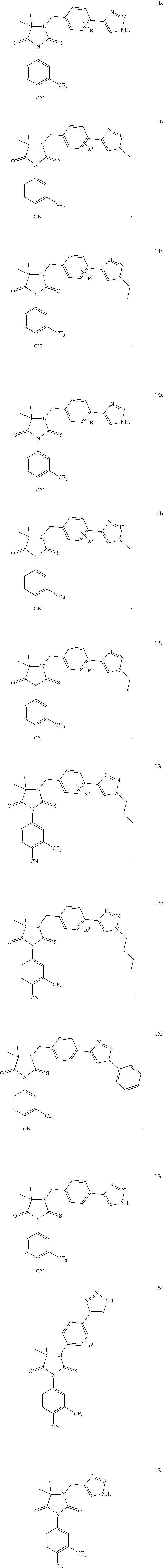 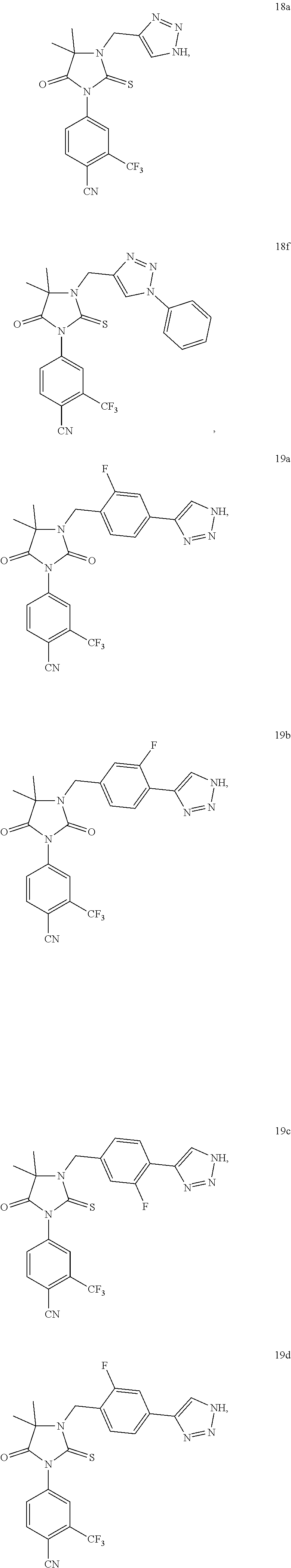 [SMILES: CC1(C)C(=O)N(c2ccc(C#N)c(C(F)(F)F)c2)C(=S)N1Cc1c[nH]nn1 ; CC1(C)C(=O)N(c2ccc(C#N)c(C(F)(F)F)c2)C(=S)N1Cc1cn(-c2ccccc2)nn1 ; CC1(C)C(=O)N(c2ccc(C#N)c(C(F)(F)F)c2)C(=O)N1Cc1ccc(-c2c[nH]nn2)cc1F ; CC1(C)C(=O)N(c2ccc(C#N)c(C(F)(F)F)c2)C(=O)N1Cc1ccc(-c2c[nH]nn2)c(F)c1 ; CC1(C)C(=O)N(c2ccc(C#N)c(C(F)(F)F)c2)C(=S)N1Cc1ccc(-c2c[nH]nn2)c(F)c1 ; CC1(C)C(=O)N(c2ccc(C#N)c(C(F)(F)F)c2)C(=S)N1Cc1ccc(-c2c[nH]nn2)cc1F] 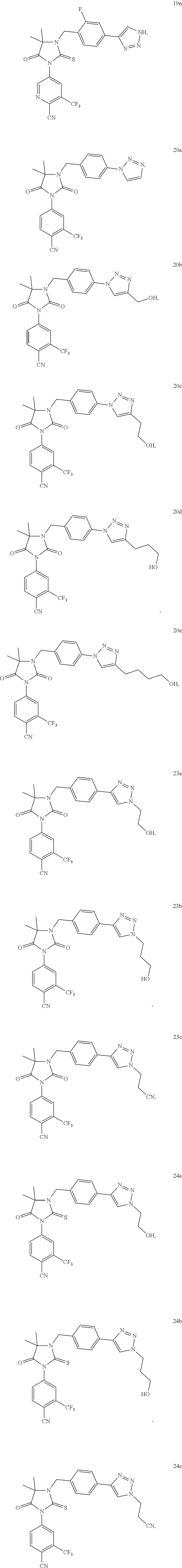 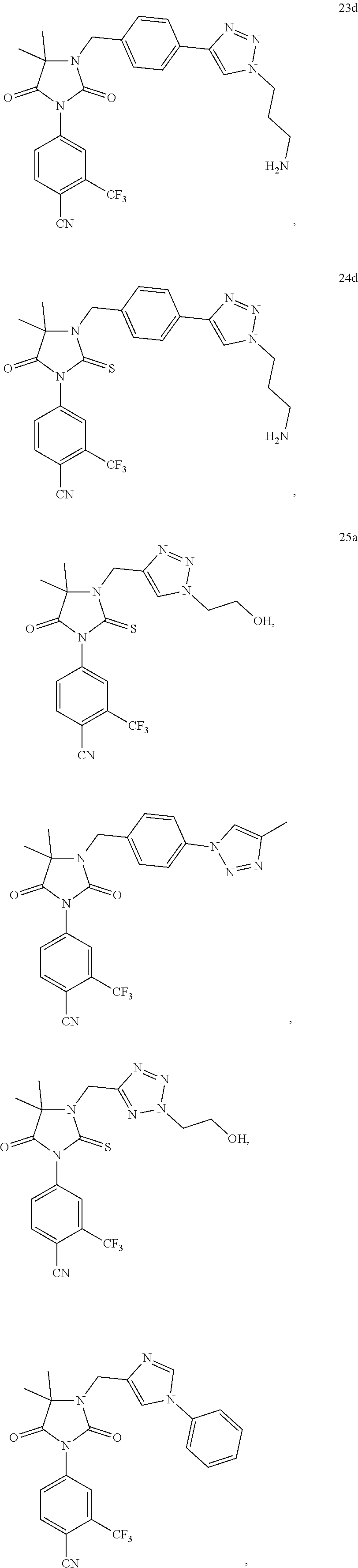 [SMILES: CC1(C)C(=O)N(c2ccc(C#N)c(C(F)(F)F)c2)C(=O)N1Cc1ccc(-c2cn(CCCN)nn2)cc1 ; CC1(C)C(=O)N(c2ccc(C#N)c(C(F)(F)F)c2)C(=S)N1Cc1ccc(-c2cn(CCCN)nn2)cc1 ; CC1(C)C(=O)N(c2ccc(C#N)c(C(F)(F)F)c2)C(=S)N1Cc1cn(CCO)nn1 ; Cc1cn(-c2ccc(CN3C(=O)N(c4ccc(C#N)c(C(F)(F)F)c4)C(=O)C3(C)C)cc2)nn1 ; CC1(C)C(=O)N(c2ccc(C#N)c(C(F)(F)F)c2)C(=S)N1Cc1nnn(CCO)n1 ; CC1(C)C(=O)N(c2ccc(C#N)c(C(F)(F)F)c2)C(=O)N1Cc1cn(-c2ccccc2)cn1] [chemical structure] 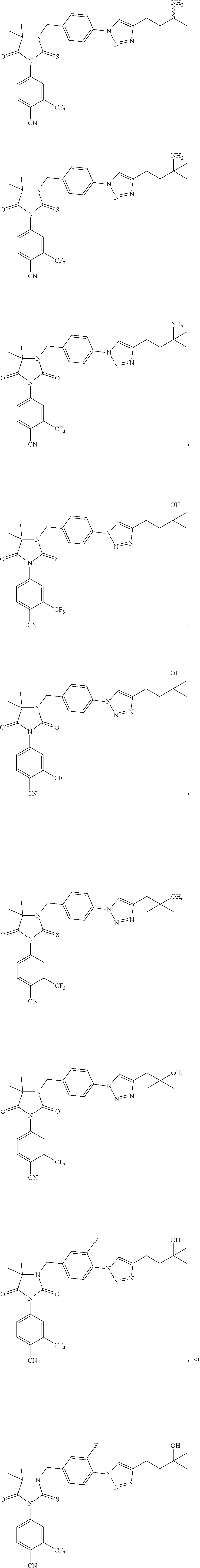
or a pharmaceutically acceptable salt thereof.

16. The compound of claim 15 that is 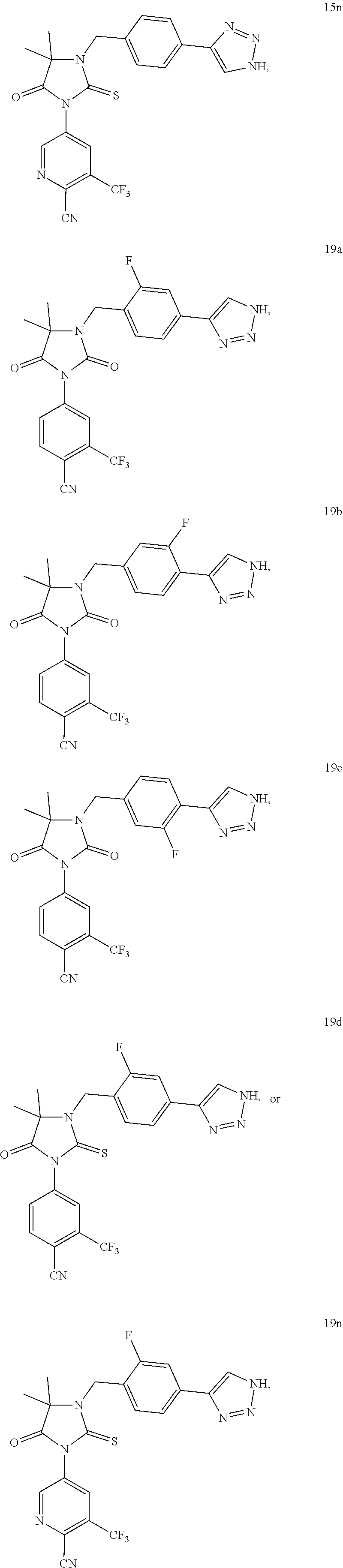 [SMILES: CC1(C)C(=O)N(c2cnc(C#N)c(C(F)(F)F)c2)C(=S)N1Cc1ccc(-c2c[nH]nn2)cc1 ; CC1(C)C(=O)N(c2ccc(C#N)c(C(F)(F)F)c2)C(=O)N1Cc1ccc(-c2c[nH]nn2)cc1F ; CC1(C)C(=O)N(c2ccc(C#N)c(C(F)(F)F)c2)C(=O)N1Cc1ccc(-c2c[nH]nn2)c(F)c1 ; CC1(C)C(=O)N(c2ccc(C#N)c(C(F)(F)F)c2)C(=O)N1Cc1ccc(-c2c[nH]nn2)c(F)c1 ; CC1(C)C(=O)N(c2ccc(C#N)c(C(F)(F)F)c2)C(=S)N1Cc1ccc(-c2c[nH]nn2)cc1F ; CC1(C)C(=O)N(c2cnc(C#N)c(C(F)(F)F)c2)C(=S)N1Cc1ccc(-c2c[nH]nn2)cc1F]
or a pharmaceutically acceptable salt thereof.

17. A pharmaceutical composition comprising the compound of claim 1, or a pharmaceutically acceptable salt thereof, and a pharmaceutically acceptable carrier.

18. A method for treating a disorder associated with androgen receptor malfunction in a subject in need thereof, the method comprising administering to the subject an effective amount of the compound of claim 1 or a pharmaceutically acceptable salt thereof.

19. The method of claim 18, wherein the disorder associated with androgen receptor malfunction is a hyperproliferative disorder.

20. The method of claim 18, wherein the disorder associated with androgen receptor malfunction is a hormone-sensitive cancer.

21. The method of claim 18, wherein the disorder associated with androgen receptor malfunction is a hormone refractory cancer.

22. The method of claim 18, wherein the disorder associated with androgen receptor malfunction is prostate cancer.
